SEQUENCE LISTING (74 sequences)

SEQ ID NO: 1 - protein, 5 aa, synthetic construct
  REGION 1..5: note = Synthetic
SYDMS

SEQ ID NO: 2 - protein, 17 aa, synthetic construct
  REGION 1..17: note = Synthetic
WISYSGGSIYYADSVKG

SEQ ID NO: 3 - protein, 12 aa, synthetic construct
  REGION 1..12: note = Synthetic
DAQRNSMREFDY

SEQ ID NO: 4 - protein, 13 aa, synthetic construct
  REGION 1..13: note = Synthetic
SGSSSNIGNNYVT

SEQ ID NO: 5 - protein, 7 aa, synthetic construct
  REGION 1..7: note = Synthetic
ADSHRPS

SEQ ID NO: 6 - protein, 11 aa, synthetic construct
  REGION 1..11: note = Synthetic
ATWDYSLSGYV

SEQ ID NO: 7 - protein, 5 aa, synthetic construct
  REGION 1..5: note = Synthetic
TFGMH

SEQ ID NO: 8 - protein, 17 aa, synthetic construct
  REGION 1..17: note = Synthetic
YITSGESPIYFTDTVKG

SEQ ID NO: 9 - protein, 10 aa, synthetic construct
  REGION 1..10: note = Synthetic
SSYYGNSMDY

SEQ ID NO: 10 - protein, 17 aa, synthetic construct
  REGION 1..17: note = Synthetic
RSSQSLLNAGNRKNYLT

SEQ ID NO: 11 - protein, 7 aa, synthetic construct
  REGION 1..7: note = Synthetic
WASTRES

SEQ ID NO: 12 - protein, 9 aa, synthetic construct
  REGION 1..9: note = Synthetic
QNAYSYPFT

SEQ ID NO: 13 - protein, 5 aa, synthetic construct
  REGION 1..5: note = Synthetic
TYPIE

SEQ ID NO: 14 - protein, 17 aa, synthetic construct
  REGION 1..17: note = Synthetic
NFHPYNDDTKYNEKFKG

SEQ ID NO: 15 - protein, 11 aa, synthetic construct
  REGION 1..11: note = Synthetic
RAYGYPYAMDY

SEQ ID NO: 16 - protein, 17 aa, synthetic construct
  REGION 1..17: note = Synthetic
KSSQSLLNAGNQKNYLT

SEQ ID NO: 17 - protein, 7 aa, synthetic construct
  REGION 1..7: note = Synthetic
RASSRES

SEQ ID NO: 18 - protein, 9 aa, synthetic construct
  REGION 1..9: note = Synthetic
QNDYIYPYT

SEQ ID NO: 19 - protein, 5 aa, synthetic construct
  REGION 1..5: note = Synthetic
GYIIQ

SEQ ID NO: 20 - protein, 17 aa, synthetic construct
  REGION 1..17: note = Synthetic
FINPYNDDTKYNEQFKG

SEQ ID NO: 21 - protein, 9 aa, synthetic construct
  REGION 1..9: note = Synthetic
AYFGNAFAY

SEQ ID NO: 22 - protein, 17 aa, synthetic construct
  REGION 1..17: note = Synthetic
KSSQSLLNAGNQKNYLT

SEQ ID NO: 23 - protein, 9 aa, synthetic construct
  REGION 1..9: note = Synthetic
QNAYYFPFT

SEQ ID NO: 24 - protein, 121 aa, synthetic construct
  REGION 1..121: note = Synthetic
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 25 - protein, 110 aa, synthetic construct
  REGION 1..110: note = Synthetic
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 26 - protein, 119 aa, synthetic construct
  REGION 1..119: note = Synthetic
EVQLVESGGGLVQPGGSLRLSCAASGFTFSTFGMHWVRQAPGKGLEWVSYITSGESPIYFTDTVKGRFTISRDNAKNSLYLQMNSLRAEDTAVYYCARSSYYGNSMDYWGQGTLVTVSS

SEQ ID NO: 27 - protein, 113 aa, synthetic construct
  REGION 1..113: note = Synthetic
DIVMTQSPDSLAVSLGERATINCRSSQSLLNAGNRKNYLTWYQQKPGQPPKLLIYWASTRESGVPDRFSGSGSGTDFTLTISSLQAEDVAVYYCQNAYSYPFTFGGGTKLEIK

SEQ ID NO: 28 - protein, 120 aa, synthetic construct
  REGION 1..120: note = Synthetic
QVQLVQSGAEVKKPGASVKVSCKASGYTFTTYPIEWVRQAPGQRLEWMGNFHPYNDDTKYNEKFKGRVTITRDTSASTAYMELSSLRSEDTAVYYCARRAYGYPYAMDYWGQGTLVTVSS

SEQ ID NO: 29 - protein, 113 aa, synthetic construct
  REGION 1..113: note = Synthetic
DIVMTQSPDSLAVSLGERATINCKSSQSLLNAGNQKNYLTWYQQKPGQPPKLLIYRASSRESGVPDRFSGSGSGTDFTLTISSLQAEDVAVYYCQNDYIYPYTFGGGTKLEIK

SEQ ID NO: 30 - protein, 118 aa, synthetic construct
  REGION 1..118: note = Synthetic
QVQLVQSGAEVKKPGASVKVSCKASGYTFTGYIIQWVRQAPGQRLEWMGFINPYNDDTKYNEQFKGRVTITRDTSASTAYMELSSLRSEDTAVYYCARAYFGNAFAYWGQGTLVTVSS

SEQ ID NO: 31 - protein, 113 aa, synthetic construct
  REGION 1..113: note = Synthetic
DIVMTQSPDSLAVSLGERATINCKSSQSLLNAGNQKNYLTWYQQKPGQPPKLLIYWASTRESGVPDRFSGSGSGTDFTLTISSLQAEDVAVYYCQNAYYFPFTFGGGTKLEIK

SEQ ID NO: 32 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 107 aa, synthetic construct [sequence omitted]

SEQ ID NO: 34 - protein, 15 aa, synthetic construct
  REGION 1..15: note = Synthetic
GGGGSGGGGSGGGGS

SEQ ID NO: 35 - protein, 20 aa, synthetic construct
  REGION 1..20: note = Synthetic
GGGGSGGGGSGGGGSGGGGS

SEQ ID NO: 36 - protein, 103 aa, synthetic construct
  REGION 1..103: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 37 - protein, 106 aa, synthetic construct
  REGION 1..106: note = Synthetic
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 38 - protein, 64 aa, synthetic construct
  REGION 1..64: note = Synthetic
GSGSGSGSGSGSGSGSGSGGGGSGGGGSGGGGSGGGGSGGGGSGGGGSGSGSGSGSGSGSGSGS

SEQ ID NO: 39 - protein, 255 aa, synthetic construct
  REGION 1..255: note = Synthetic
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 40 - protein, 715 aa, synthetic construct
  REGION 1..715: note = Synthetic
EVQLVESGGGLVQPGGSLRLSCAASGFTFSTFGMHWVRQAPGKGLEWVSYITSGESPIYFTDTVKGRFTISRDNAKNSLYLQMNSLRAEDTAVYYCARSSYYGNSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 41 - protein, 220 aa, synthetic construct
  REGION 1..220: note = Synthetic
DIVMTQSPDSLAVSLGERATINCRSSQSLLNAGNRKNYLTWYQQKPGQPPKLLIYWASTRESGVPDRFSGSGSGTDFTLTISSLQAEDVAVYYCQNAYSYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 716 aa, synthetic construct
  REGION 1..716: note = Synthetic
QVQLVQSGAEVKKPGASVKVSCKASGYTFTTYPIEWVRQAPGQRLEWMGNFHPYNDDTKYNEKFKGRVTITRDTSASTAYMELSSLRSEDTAVYYCARRAYGYPYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 43 - protein, 220 aa, synthetic construct
  REGION 1..220: note = Synthetic
DIVMTQSPDSLAVSLGERATINCKSSQSLLNAGNQKNYLTWYQQKPGQPPKLLIYRASSRESGVPDRFSGSGSGTDFTLTISSLQAEDVAVYYCQNDYIYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 714 aa, synthetic construct
  REGION 1..714: note = Synthetic
QVQLVQSGAEVKKPGASVKVSCKASGYTFTGYIIQWVRQAPGQRLEWMGFINPYNDDTKYNEQFKGRVTITRDTSASTAYMELSSLRSEDTAVYYCARAYFGNAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 45 - protein, 220 aa, synthetic construct
  REGION 1..220: note = Synthetic
DIVMTQSPDSLAVSLGERATINCKSSQSLLNAGNQKNYLTWYQQKPGQPPKLLIYWASTRESGVPDRFSGSGSGTDFTLTISSLQAEDVAVYYCQNAYYFPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 121 aa, synthetic construct
  REGION 1..121: note = Synthetic
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDGQRNSMREFDYWGQGTLVTVSS

SEQ ID NO: 47 - protein, 121 aa, synthetic construct
  REGION 1..121: note = Synthetic
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRNSMREFDYWGQGTLVTVSS

SEQ ID NO: 48 - protein, 121 aa, synthetic construct
  REGION 1..121: note = Synthetic
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRQSMREFDYWGQGTLVTVSS

SEQ ID NO: 49 - protein, 121 aa, synthetic construct
  REGION 1..121: note = Synthetic
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDGQRQSMREFDYWGQGTLVTVSS

SEQ ID NO: 50 - protein, 121 aa, synthetic construct
  REGION 1..121: note = Synthetic
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRNAMREFDYWGQGTLVTVSS

SEQ ID NO: 51 - protein, 127 aa, synthetic construct
  REGION 1..127: note = Synthetic
EVQLLESGGGLVQPGGSLRLSCAASGFTFSGYDMSWVRQAPGKGLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 52 - protein, 110 aa, synthetic construct
  REGION 1..110: note = Synthetic
QSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGGGTKLTVL

SEQ ID NO: 53 - protein, 110 aa, synthetic construct
  REGION 1..110: note = Synthetic
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGGGTKLTVL

SEQ ID NO: 54 - protein, 973 aa, synthetic construct
  REGION 1..973: note = Synthetic
EVQLVESGGGLVQPGGSLRLSCAASGFTFSTFGMHWVRQAPGKGLEWVSYITSGESPIYFTDTVKGRFTISRDNAKNSLYLQMNSLRAEDTAVYYCARSSYYGNSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSGSGSGSGSGSGSGSGSGSGGGGSGGGGSGGGGSGGGGSGGGGSGGGGSGSGSGSGSGSGSGSGSEVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRNSMREFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 968 aa, synthetic construct
  REGION 1..968: note = Synthetic
EVQLVESGGGLVQPGGSLRLSCAASGFTFSTFGMHWVRQAPGKGLEWVSYITSGESPIYFTDTVKGRFTISRDNAKNSLYLQMNSLRAEDTAVYYCARSSYYGNSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSGSGSGSGSGSGSGSGSGSGGGGSGGGGSGGGGSGGGGSGGGGSGGGGSGSGSGSGSGSGSGSGSEVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRNSMREFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 56 - protein, 12 aa, synthetic construct
  REGION 1..12: note = Synthetic
DGQRNSMREFDY

SEQ ID NO: 57 - protein, 12 aa, synthetic construct
  REGION 1..12: note = Synthetic
DAQRQSMREFDY

SEQ ID NO: 58 - protein, 12 aa, synthetic construct
  REGION 1..12: note = Synthetic
DGQRQSMREFDY

SEQ ID NO: 59 - protein, 12 aa, synthetic construct
  REGION 1..12: note = Synthetic
DAQRNAMREFDY

SEQ ID NO: 60 - protein, 5 aa, synthetic construct
  REGION 1..5: note = Synthetic
GYDMS

SEQ ID NO: 61 - protein, 17 aa, synthetic construct
  REGION 1..17: note = Synthetic
VIYPDDGNTYYADSVKG

SEQ ID NO: 62 - protein, 18 aa, synthetic construct
  REGION 1..18: note = Synthetic
HGGQKPTTKSSSAYGMDG

SEQ ID NO: 63 - protein, 121 aa, synthetic construct
  REGION 1..121: note = Synthetic
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDGQRNSMREFDYWGQGTLVTVSS

SEQ ID NO: 64 - protein, 121 aa, synthetic construct; identical to SEQ ID NO: 24
  REGION 1..121: note = Synthetic

SEQ ID NO: 65 - protein, 121 aa, synthetic construct; identical to SEQ ID NO: 24
  REGION 1..121: note = Synthetic

SEQ ID NO: 66 - protein, 121 aa, synthetic construct
  REGION 1..121: note = Synthetic
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRQSMREFDYWGQGTLVTVSS

SEQ ID NO: 67 - protein, 127 aa, synthetic construct
  REGION 1..127: note = Synthetic
EVQLLESGGGLVQPGGSLRLSCAASGFTFSGYDMSWVRQAPGKCLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 68 - protein, 127 aa, synthetic construct
  REGION 1..127: note = Synthetic
EVQLLESGGGLVQPGGSLRLSCAASGFTFSGYDMSWVRQAPGKCLEWVSVIYPDDGNTYYADSVKGRFTISRDNSKNTLYLQMNSLRAEDTAVYYCAKHGGQKPTTKSSSAYGMDGWGQGTLVTVSS

SEQ ID NO: 69 - protein, 110 aa, synthetic construct
  REGION 1..110: note = Synthetic
QSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 70 - protein, 110 aa, synthetic construct; identical to SEQ ID NO: 69
  REGION 1..110: note = Synthetic

SEQ ID NO: 71 - protein, 110 aa, synthetic construct; identical to SEQ ID NO: 25
  REGION 1..110: note = Synthetic

SEQ ID NO: 72 - protein, 110 aa, synthetic construct; identical to SEQ ID NO: 25
  REGION 1..110: note = Synthetic

SEQ ID NO: 73 - protein, 110 aa, synthetic construct; identical to SEQ ID NO: 69
  REGION 1..110: note = Synthetic

SEQ ID NO: 74 - protein, 110 aa, synthetic construct; identical to SEQ ID NO: 25
  REGION 1..110: note = Synthetic

(19) United States
(12) Patent Application Publication
(10) Pub. No.: US 2026/0184803 A1
(43) Pub. Date: Jul. 2, 2026

(54) ANTI-CLAUDIN 18.2 AND ANTI-4-1BB BISPECIFIC ANTIBODIES AND USES THEREOF

(71) Applicants: I-MAB Biopharma US Limited, Rockville, MD (US); ABL BIO INC., Seongnam-si Gyeonggi-Do (KR)

(72) Inventors: Wenqing JIANG, Shanghai (CN); Lei FANG, Shanghai (CN); Zhengyi WANG, Shanghai (CN); Bingshi GUO, Shanghai (CN); Eunyoung PARK, Seongnam-si Gyeonggi-Do (KR); Eunsil SUNG, Seongnam-si Gyeonggi-Do (KR); Byungje SUNG, Seongnam-si Gyeonggi-Do (KR)

(21) Appl. No.: 19/561,007

(22) Filed: Mar. 9, 2026

Related U.S. Application Data
(63) Continuation of application No. 17/683,869, filed on Mar. 1, 2022, now Pat. No. 12,577,313.
Application No. 17/683,869 is a division of application No. 17/286,437, now Pat. No. 11,261,259.

(30) Foreign Application Priority Data
Aug. 12, 2019 (WO) PCT/CN2019/100162
Sep. 5, 2019 (WO) PCT/CN2019/104508
Jan. 14, 2020 (WO) PCT/CN2020/071954
Apr. 30, 2020 (WO) PCT/CN2020/087968

(51) Int. Cl. C07K 16/28; A61K 39/00; A61P 35/00

(52) U.S. Cl. CPC C07K 16/2878; A61P 35/00; C07K 16/28; A61K 2039/505; C07K 2317/31; C07K 2317/52; C07K 2317/622; C07K 2317/92

(57) ABSTRACT

Provided are bispecific and multi-specific antibodies that target both claudin 18.2 (CLDN18.2) and 4-1BB. These antibodies, in the absence of CLDN18.2-expressing cells, can bind to 4-1BB but are unable to activate 4-1BB signaling. In the presence of CLDN18.2-expressing cells, however, these antibodies can trigger CLDN18.2-dependent 4-1BB signaling, leading to potent immune response to the CLDN18.2-expressing tumor cells.

### CROSS REFERENCE TO RELATED APPLICATIONS

This application is a continuation of U.S. application Ser. No. 17/683,869, filed Mar. 1, 2022, which is a divisional of U.S. application Ser. No. 17/286,437, filed Apr. 16, 2021, which claims the benefit of PCT Application No. PCT/CN2020/108707, filed Aug. 12, 2020, which claims the benefit of PCT Application Serial No. PCT/CN2019/100162, filed Aug. 12, 2019, No. PCT/CN2019/104508, filed Sep. 5, 2019, No. PCT/CN2020/071954, filed Jan. 14, 2020, and No. PCT/CN2020/087968, filed Apr. 30, 2020, the content of each of which is hereby incorporated by reference in its entirety.

### REFERENCE TO SEQUENCE LISTING SUBMITTED ELECTRONICALLY

This application contains a sequence listing, which is submitted electronically via EFS-Web as an xml formatted sequence listing with a file name of “077198-3US3-SequenceListing.xml” creation date of Feb. 25, 2026 and having a size of 101,134 bytes. The sequence listing submitted via EFS-Web is part of the specification and is herein incorporated by reference in its entirety.

### BACKGROUND

Claudins are a family of proteins that form the important components of the tight cell junctions. Claudin-18 splice variant 2 (CLDN18.2) is a gastric-specific membrane protein. In the healthy tissue, CLDN18.2 is restrictly expressed in the short-lived differentiated cells of gastric mucosa as a component of tight junction with limited accessibility of antibody treatment. However, it was ectopically expressed at significant levels in a variety of primary lesion and metastases of epithelial tumor entities, including gastric, pancreatic, esophageal, and lung adenocarcinoma cells.

4-1BB (CD137, tumor necrosis factor receptor superfamily 9) is a member of TNF-receptor superfamily (TNFRSF) and is a costimulatory molecule which is expressed following the activation of immune cells, both innate and adaptive immune cells. 4-1BB plays an important role in modulating the activity of various immune cells. 4-1BB agonists enhance immune cell proliferation, survival, secretion of cytokines and cytolytic activity CD8 T cells. Many other studies showed that activation of 4-1BB enhances immune response to eliminate tumors in mice. Therefore, it was suggested that 4-1BB is a promising target molecule in cancer immunology.

### SUMMARY

Provided are bispecific and multi-specific antibodies that target both claudin 18.2 (CLDN18.2) and 4-1BB. In some embodiments, the antibodies of the present technology include a “conditional agonist” anti-4-1BB portion which, without the anti-CLDN18.2 portion binding to CLDN18.2 proteins expressed on a cell, cannot activate 4-1BB signaling.

4-1BB signaling activation is the expected mechanism for agonist antibodies, such as utomilumab (PF-05082566) and urelumab (BMS-663513). The anti-4-1BB portions of the presently disclosed antibodies, however, do not require such an activity. Actually, it is preferred that the anti-4-1BB portions of the present antibodies are not capable of independently activating 4-1BB in the absence of CLDN18.2 binding. As the experimental examples demonstrated, interestingly, when the anti-CLDN18.2 portion binds to CLDN18.2 proteins on a cell, such CLDN18.2 binding can trigger 4-1BB signaling activation.

Compared to the known anti-4-1BB agonist antibodies which are commonly associated with dose-limiting on-target toxicities, the antibodies of the present disclosure are contemplated to be much safer. In a tissue, such as liver, wherein CLDN18.2 is not expressed, the present antibodies are not expected to trigger cytotoxic immune response as they cannot activate 4-1BB signaling. In a tumor tissue wherein CLDN18.2 is expressed and/or accessible, by contrast, the present antibodies can initiate potent immune response to the tumor cells. Accordingly, unlike those anti-4-1BB antibodies currently being developed clinically which suffer on-target/inherent toxicities, the presently disclosed antibodies can be potent and safe at the same time in treating cancer.

### BRIEF DESCRIPTION OF THE DRAWINGS

FIG. 1A-C illustrate three different bispecific formats tested in the present disclosure.

FIG. 2A-D present ELISA results for 4-1BB and CLDN18.2 binding.

FIG. 3 shows the cell-based 4-1BB binding.

FIG. 4A-C show the binding results for CLDN18.2.

FIG. 5A-D show CLDN18.2-dependent 4-1BB signaling activation.

FIG. 6A-E show PBMC response in the presence of CLDN18.2 expressing cells.

FIG. 7A-D show CD8+T response in the presence of CLDN18.2 expressing cells.

FIG. 8A-B show in vivo efficacy of anti-CLDN18.2-4-1BB antibody in syngeneic mouse model and ex vivo analysis of its impact on tumor infiltrating lymphocytes.

FIG. 9A-C show in vivo efficacy of C-1A10 bispecific antibody in syngeneic mouse model.

FIG. 10 shows dose-dependent anti-tumor efficacy of C-1A10 and D-1A10 in syngeneic mouse model.

FIG. 11A-D show pharmacokinetics and pharmacodynamics relation of D-1A10 in syngeneic mouse model.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Biologically equivalent polynucleotides are those having the above-noted specified percent homology and encoding a polypeptide having the same or similar biological activity.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′) 2, F (ab) 2, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (□, □, □, □) with some subclasses among them (e.g., □1-□□4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgAQ1 and IgA2) or subclass of immunoglobulin molecule.

Light chains are classified as either kappa or lambda (0, 0). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt □-sheet conformation and the CDRs form loops which connect, and in some cases form part of, the □-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| | Kabat | Chothia |
|---|---|---|
| CDR-H1 | 31-35 | 26-32 |
| CDR-H2 | 50-65 | 52-58 |
| CDR-H3 | 95-102 | 95-102 |
| CDR-L1 | 24-34 | 26-32 |
| CDR-L2 | 50-56 | 50-52 |
| CDR-L3 | 89-97 | 91-96 |

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CHI domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG molecule and a hinge region derived from an IgG3 molecule. In another example, a heavy chain constant region can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CHI domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al., U.S. Dept. of Health and Human Services, “Sequences of Proteins of Immunological Interest” (1983). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol 161:4083 (1998)).

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Anti-Claudin 18.2 Anti-4-1BB Antibodies

4-1BB is an inducible costimulatory receptor expressed on activated T and natural killer (NK) cells. 4-1BB trimer clustering by 4-1BB ligand (41BBL) trimer on T cells triggers a signaling cascade that results in upregulation of antiapoptotic molecules, cytokine secretion, and enhanced effector function. On NK cells, 4-1BB signaling can increase antibody-dependent cell-mediated cytotoxicity. Agonistic monoclonal antibodies targeting 4-1BB have been developed to harness 4-1BB signaling for cancer immunotherapy. Preclinical results in a variety of induced and spontaneous tumor models suggest that targeting 4-1BB with agonist antibodies can lead to tumor clearance and durable antitumor immunity.

Two agonist antibodies, urelumab and utomilumab, are currently undergoing clinical trials. Urelumab has strong efficacy but showed inflammatory liver toxicities. The liver toxicity appears to be on-target, making it difficult to be separated from efficacy. Utomilumab is relatively safer than urelumab, but is also less effective.

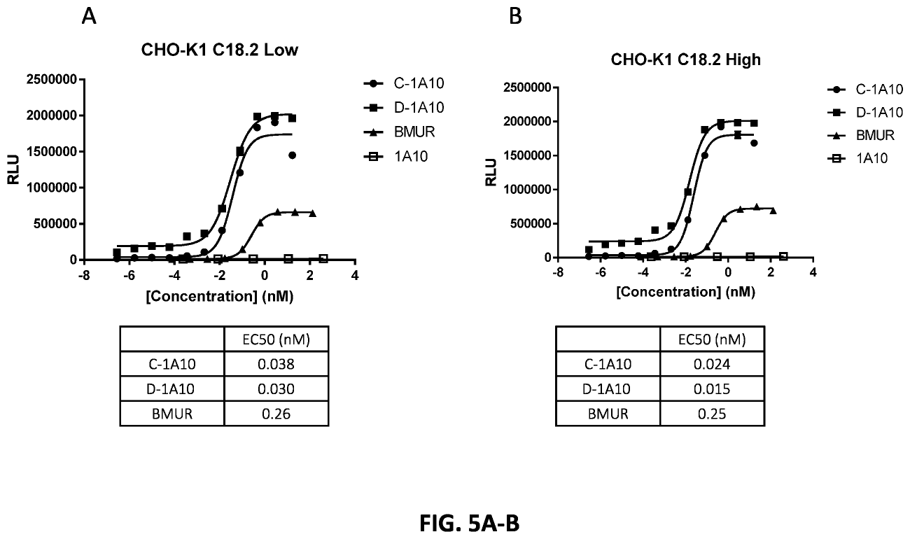

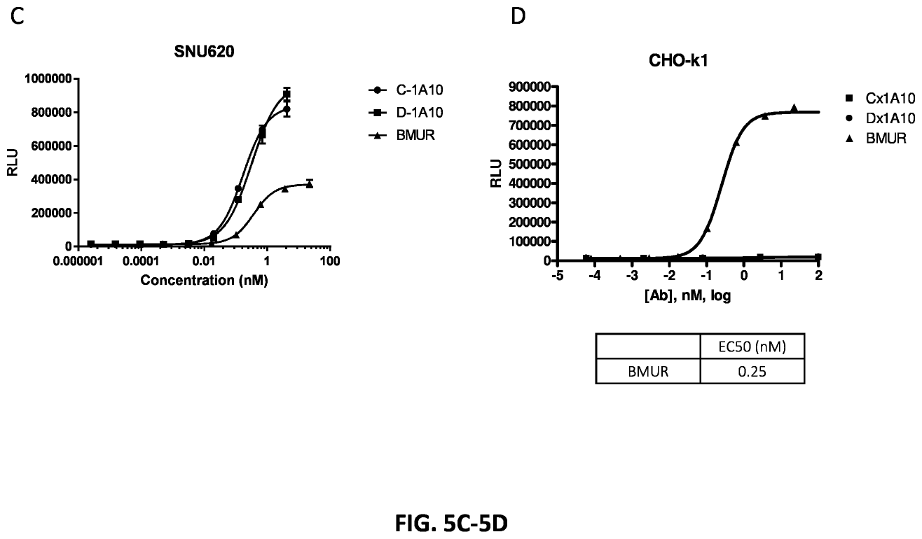

The present experimental example tested a couple of anti-4-1BB antibodies which were specifically selected for their incapability to activate 4-1BB signaling independently. In their monospecific forms, they can bind to 4-1BB alone or 4-1BB on the cell surface. Their binding of 4-1BB on the cell surface, however, does not lead to 4-1BB signaling activation (see, e.g., Example 3 and FIG. 5).

Interestingly, when the binding fragments of one of these conditional agonist 4-1BB antibodies, 1A10, was incorporated into a bispecific antibody that further has an anti-CLDN18.2 portion, the resulting bispecific antibody was able to efficiently activate 4-1BB signaling in a CLDN18.2-binding-dependent manner (see Example 3 and FIG. 5). It is worth noting that these tested bispecific antibodies employed an N297A IgG1 Fc to disable FcγR-medicated 4-1BB agonism. Therefore, the 4-1BB signaling activation can only be attributed to the CLDN18.2 binding.

The on-target toxicity of the anti-4-1BB agonist antibody urelumab is attributed to the antibody' lack of selectivity in 4-1BB agonism. The antibodies of the present technology, however, can be readily recognized for their ability to overcome this limitation. In a tissue, such as liver, where CLDN18.2 is not expressed or accessible, the antibodies would be safe as they cannot activate 4-1BB-mediated cytotoxicity. In a tumor tissue wherein CLDN18.2 is overexpressed or accessible, by contrast, the antibodies undergo CLDN18.2 binding-dependent 4-1BB signaling activation, leading to 4-1BB-mediated immune cell activation, thereby treating the tumor.

In accordance with one embodiment of the present disclosure, therefore, provided is an antibody that includes an anti-claudin 18.2 (CLDN18.2) unit having binding specificity to a CLDN18.2 protein, and an anti-4-1BB unit having binding specificity to a 4-1BB protein. In a preferred embodiment, the anti-4-1BB unit is incapable of activating 4-1BB signaling upon binding to a 4-1BB protein, in the absence of the anti-CLDN18.2 unit binding to a CLDN18.2 protein.

The lack of 4-1BB agonism of the anti-4-1BB portion can be achieved by many different means. It is suggested that 4-1BB clustering on the cell surface is required for its activation. In some embodiments, therefore, the binding of the anti-4-1BB unit to 4-1BB proteins on a cell does not result in clustering of the 4-1BB proteins, in the absence of the anti-CLDN18.2 unit binding to a CLDN18.2 protein.

The 4-1BB protein has four extracellular cysteine-rich pseudo repeats (CRD) domains, CRD1, CRD2, CRD3 and CRD4 (see the amino acid sequence and the CRD regions in the table below). Urelumab binds to the N-terminal CDR1, and activates 4-1BB clustering in a 4-1BB ligand (4-1BBL)-dependently manner. In some embodiments, the anti-4-1BB unit of the presently disclosed antibodies does not bind to CDR1. In some embodiments, the anti-4-1BB unit of the presently disclosed antibodies binds to CDR2. In some embodiments, the anti-4-1BB unit of the presently disclosed antibodies binds to CDR3. In some embodiments, the anti-4-1BB unit of the presently disclosed antibodies binds to CDR4.

Sequence and CRD Annotation (SEQ ID NO: 39)

| | |
|---|---|
| Human 4-1BB | 1 MGNSCYNIVA TLLLVLNFER TRSLQDPCSN CPAGTFCDNN RNQICSPCPP |
| (NP_001552.2) | Signal peptide CRD1 |
| | 51 NSFSSAGGQR TCDICRQCKG VFRTRKECSS TSNAECDCTP |
| | GFHCLGAGCS |
| | CRD2 CRD3 |
| | 101 MCEQDCKQGQ ELTKKGCKDC CFGTFNDQKR GICRPWTNCS |
| | LDGKSVLVNG |
| | CRD4 |
| | 151 TKERDVVCGP SPADLSPGAS SVTPPAPARE PGHSPQIISF FLALTSTALL |
| | 201 FLLFFLTLRF SVVKRGRKKL LYIFKQPFMR PVQTTQEEDG CSCRFPEEEE |
| | 251 GGCEL |

In some instances, 4-1BB clustering may be mediated through the effector function of an anti-4-1BB antibody. In some embodiments, therefore, an antibody of the present disclosure has an Fc fragment that has reduced or no effector function. Such effort functions, in some embodiments, antibody-dependent cell-mediated cytotoxicity (ADCC), complement-dependent cytotoxicity (CDC), or antibody-dependent cellular phagocytosis (ADCP).

The effector function of an antibody can be altered using techniques known in the art. In some embodiments, the Fc fragment of the antibody is mutated, or manufactured, in a manner to reduce or eliminate its binding to FcγR. In some embodiments, the Fc fragment has reduced or no binding to FcγRI (CD64); in some embodiments, the Fc fragment has reduced or no binding to FcγRIIA (CD32); in some embodiments, the Fc fragment has reduced or no binding to FcγRIIB (CD32); in some embodiments, the Fc fragment has reduced or no binding to FcγRIIIA (CD16a); and in some embodiments, the Fc fragment has reduced or no binding to FcγRIIIB (CD16b). In some embodiments, the Fc fragment has reduced or no binding to C1q (first subcomponent of the C1 complex).

In some embodiments, the Fc fragment includes one or more mutations that reduce or eliminate the FcγR or C1q binding of the Fc fragment. Non-limiting examples of such mutations include the L235E mutation in an IgG1 Fc fragment, the L234A and/or L235A mutation in an IgG1 Fc fragment, the P329G or P329A mutation in an IgG1 Fc fragment, the F234A and/or L235A or L235E mutation in an IgG4 Fc fragment, the H268Q, V309L, A330S, and/or P331S mutation in an IgG2 Fc fragment, and the V234A, G237A, P238S, H268A, V309L, A330S, and/or P331S mutation in an IgG2 Fc fragment (EU numbering).

The effector function of an antibody can also be reduced by decreasing or inhibiting glycosylation of the Fc fragment (e.g., an aglycosylated Fc fragment). Such decrease or inhibition can be achieved, in some embodiments, by using a cell line that is incapable to glycosylate the antibody. In some embodiments, the Fc fragment is mutated.

Non-limiting examples of such mutations include a mutation at N297 (such as N297A, N297G, and N297Q) (EU numbering). In some embodiments, the mutation is N297A.

### Antibody Formats and Example Sequences

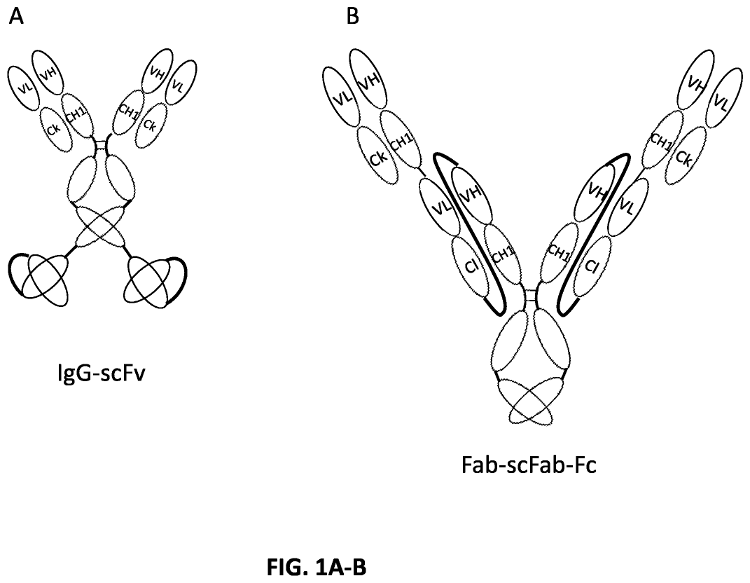

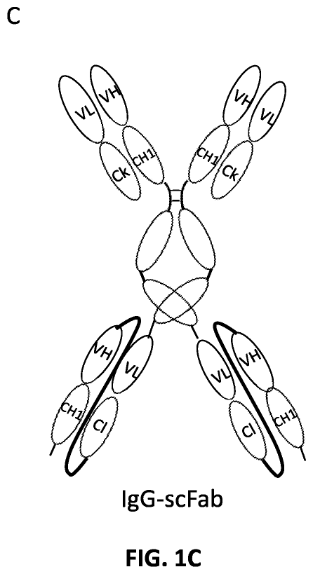

In the experimental examples, three different bispecific antibody formats were tested. Among them, the format of FIG. 1A exhibited higher activity than those of FIG. 1B and FIG. 1C. These data suggest that a suitable format for the antibodies of the present disclosure may employ a Fab fragment for the anti-CLDN18.2 portion. In some embodiments, the format includes a single chain fragment (scFv) for the anti-4-1BB portion. In some embodiments, the anti-4-1BB portion is not located between the Fab and a Fc fragment.

In some embodiments, the anti-CLDN18.2 unit include a Fab fragment, and preferably a pair of the Fab fragment. In some embodiments, the anti-4-1BB unit includes a Fab fragment, preferably a pair of the Fab fragments. In some embodiments, the anti-4-1BB unit includes a scFv, preferably a pair of the scFv fragments.

In some embodiments, the anti-CLDN18.2 unit is located at the N-terminal side of the anti-4-1BB unit. In some embodiments, a Fc fragment is placed between the anti-CLDN18.2 unit and the anti-4-1BB unit.

Example CDR sequence and VH/VL sequences are also provided for the anti-CLDN18.2 unit and the anti-4-1BB unit. In some embodiments, the anti-4-1BB unit comprises a heavy chain variable region (VH) comprising a CDRH1, a CDRH2, and a CDRH3, and a light chain variable region (VL) comprising a CDRL1, a CDRL2 and a CDRL3, wherein: the CDRH1 comprises the amino acid sequence of SEQ ID NO:1, or an amino acid sequence having one or two amino acid substitution from SEQ ID NO:1; the CDRH2 comprises the amino acid sequence of SEQ ID NO:2, or an amino acid sequence having one or two amino acid substitution from SEQ ID NO:2; the CDRH3 comprises the amino acid sequence of SEQ ID NO: 3, 56, 57, 58, or 59, or an amino acid sequence having one or two amino acid substitution from SEQ ID NO: 3, 56, 57, 58, or 59; the CDRL1 comprises the amino acid sequence of SEQ ID NO:4 or 60, or an amino acid sequence having one or two amino acid substitution from SEQ ID NO:4 or 60; the CDRL2 comprises the amino acid sequence of SEQ ID NO:5 or 61, or an amino acid sequence having one or two amino acid substitution from SEQ ID NO:5 or 61; and the CDRL3 comprises the amino acid sequence of SEQ ID NO:6 or 62, or an amino acid sequence having one or two amino acid substitution from SEQ ID NO:6 or 62.

TABLE A
CDR Sequences of an Example Anti-4-1BB Unit

| | Sequence | SEQ ID NO: |
|---|---|---|
| CDRH1 | SYDMS | 1 |
| CDRH2 | WSYSGGSIYYADSVKG | 2 |
| CDRH3 | DAQRNSMREFDY, | 3 |
| | DGQRNSMREFDY, | 56 |
| | DAQRQSMREFDY, | 57 |
| | DGQRQSMREFDY, or | 58 |
| | DAQRNAMREFDY | 59 |
| CDRL1 | SGSSSNIGNNYVT | 4 |
| CDRL2 | ADSHRPS | 5 |
| CDRL3 | ATWDYSLSGYV | 6 |
| | Sequence | SEQ ID NO: |
| CDRH1 | SYDMS | 1 |
| CDRH2 | WSYSGGSIYYADSVKG | 2 |
| CDRH3 | DAQRNSMREFDY, | 3 |
| | DGQRNSMREFDY, | 56 |
| | DAQRQSMREFDY, | 57 |
| | DGQRQSMREFDY, or | 58 |
| | DAQRNAMREFDY | 59 |
| CDRL1 | GYDMS | 60 |
| CDRL2 | VIYPDDGNTYYADSVKG | 61 |
| CDRL3 | HGGQKPTTKSSSAYGMDG | 62 |

In some embodiments, the CDRH1 comprises the amino acid sequence of SEQ ID NO: 1; the CDRH2 comprises the amino acid sequence of SEQ ID NO:2; the CDRH3 comprises the amino acid sequence of SEQ ID NO: 3, 56, 57, 58, or 59; the CDRL1 comprises the amino acid sequence of SEQ ID NO:4 or 60; the CDRL2 comprises the amino acid sequence of SEQ ID NO:5 or 61; and the CDRL3 comprises the amino acid sequence of SEQ ID NO:6 or 62.

In some embodiments, the anti-4-1BB unit comprises a heavy chain variable region (VH) comprising an amino acid sequence selected from the group consisting of SEQ ID NO: 24, 46-51 and 63-69 and a light chain variable region (VL) comprising an amino acid sequence selected from the group consisting of SEQ ID NO:25, 52-53 and 70-74.

TABLE B
VH/VL Sequences of an
Example Anti-4-1BB Unit

| | |
|---|---|
| 1A10 VH | EVQLLESGGGLVQPGGSLRLSCAASGF |
| | TFSSYDMSWVRQAPGKCLEWVSWISYS |
| | GGSIYYADSVKGRFTISRDNSKNTLYL |
| | QMNSLRAEDTAVYYCARDAQRNSMREF |
| | DYWGQGTLVTVSS |
| | (SEQ ID NO: 24) |
| 1A10 VL | QSVLTQPPSASGTPGQRVTISCSGSSS |
| | NIGNNYVTWYQQLPGTAPKLLIYADSH |
| | RPSGVPDRFSGSKSGTSASLAISGLRS |
| | EDEADYYCATWDYSLSGYVFGCGTKLT |
| | VL |
| | (SEQ ID NO: 25) |

In some embodiments, the anti-CLDN18.2 unit comprises a heavy chain variable region (VH) comprising a CDRH1, a CDRH2, and a CDRH3, and a light chain variable region (VL) comprising a CDRL1, a CDRL2 and a CDRL3, wherein (a) the CDRH1 comprises the amino acid sequence of SEQ ID NO:7; the CDRH2 comprises the amino acid sequence of SEQ ID NO:8; the CDRH3 comprises the amino acid sequence of SEQ ID NO:9; the CDRL1 comprises the amino acid sequence of SEQ ID NO: 10; the CDRL2 comprises the amino acid sequence of SEQ ID NO:11; and the CDRL3 comprises the amino acid sequence of SEQ ID NO: 12.

In some embodiments, the anti-CLDN18.2 unit comprises a heavy chain variable region (VH) comprising a CDRH1, a CDRH2, and a CDRH3, and a light chain variable region (VL) comprising a CDRL1, a CDRL2 and a CDRL3, wherein (b) the CDRH1 comprises the amino acid sequence of SEQ ID NO:13; the CDRH2 comprises the amino acid sequence of SEQ ID NO:14; the CDRH3 comprises the amino acid sequence of SEQ ID NO: 15; the CDRL1 comprises the amino acid sequence of SEQ ID NO:16; the CDRL2 comprises the amino acid sequence of SEQ ID NO:17; and the CDRL3 comprises the amino acid sequence of SEQ ID NO:18.

In some embodiments, the anti-CLDN18.2 unit comprises a heavy chain variable region (VH) comprising a CDRH1, a CDRH2, and a CDRH3, and a light chain variable region (VL) comprising a CDRL1, a CDRL2 and a CDRL3, wherein (c) the CDRH1 comprises the amino acid sequence of SEQ ID NO:19; the CDRH2 comprises the amino acid sequence of SEQ ID NO:20; the CDRH3 comprises the amino acid sequence of SEQ ID NO: 21; the CDRL1 comprises the amino acid sequence of SEQ ID NO:22; the CDRL2 comprises the amino acid sequence of SEQ ID NO:11; and the CDRL3 comprises the amino acid sequence of SEQ ID NO:23.

TABLE C
CDR Sequences of Example
Anti-CLDN18.2 Units

| | | | SEQ |
|---|---|---|---|
| | | | ID |
| | | Sequence | NO: |
| 4F11E2 | CDRH1 | TFGMH | 7 |
| | CDRH2 | YITSGESPIYFTDTVKG | 8 |
| | CDRH3 | SSYYGNSMDY | 9 |
| | CDRL1 | RSSQSLLNAGNRKNYLT | 10 |
| | CDRL2 | WASTRES | 11 |
| | CDRL3 | QNAYSYPFT | 12 |
| 72C1B6A3 | CDRH1 | TYPIE | 13 |
| | CDRH2 | NFHPYNDDTKYNEKFKG | 14 |
| | CDRH3 | RAYGYPYAMDY | 15 |
| | CDRL1 | KSSQSLLNAGNQKNYLT | 16 |
| | CDRL2 | RASSRES | 17 |
| | CDRL3 | QNDYIYPYT | 18 |
| 120B7B2 | CDRH1 | GYIIQ | 19 |
| | CDRH2 | FINPYNDDTKYNEQFKG | 20 |
| | CDRH3 | AYFGNAFAY | 21 |
| | CDRL1 | KSSQSLLNAGNQKNYLT | 22 |
| | CDRL2 | WASTRES | 11 |
| | CDRL3 | QNAYYFPFT | 23 |

In some embodiments, the VH of the anti-CLDN18.2 unit comprises an amino acid sequence selected from the group consisting of SEQ ID NO:26, 28 and 30, and the VH of the anti-CLDN18.2 unit comprises an amino acid sequence selected from the group consisting of SEQ ID NO:27, 29 and 31.

In some embodiments, provided is a bispecific antibody adopting a format like illustrated in FIG. 1A. In some embodiments, provided is an antibody comprising two first polypeptides and two second polypeptides, wherein each first polypeptide comprises, from N-to C-terminus, a heavy chain variable region (VH), a CHI, a CH2, a CH3, and a single chain fragment (scFv) having specificity to a 4-1BB protein; wherein each second polypeptide comprises a light chain variable region (VL) and a CL; wherein each VH is paired with one of the VL and have specificity to a claudin 18.2 (CLDN18.2) protein; and wherein the scFv is incapable of activating 4-1BB signaling upon binding to a 4-1BB protein, in the absence of the VH/VL pairs binding to a CLDN18.2 protein.

The VH/VL pair here constitutes an anti-CLDN18.2 unit and the scFv constitutes an anti-4-1BB unit. The CH2-CH3/CH2-CH3 pair constitutes an Fc fragment. The various embodiments above relating to the anti-CLDN18.2 unit, the anti-4-1BB unit, and the Fc fragments may be applicable here as well.

The following table provides non-limiting examples of the first polypeptide (heavy component) and the second polypeptide (light component). In some embodiments, each of the first polypeptides comprises the amino acid sequence of SEQ ID NO:40 and each of the second polypeptides comprises the amino acid sequence of SEQ ID NO:41.

In some embodiments, each of the first polypeptides comprises the amino acid sequence of SEQ ID NO:42 and each of the second polypeptides comprises the amino acid sequence of SEQ ID NO:43.

In some embodiments, each of the first polypeptides comprises the amino acid sequence of SEQ ID NO:44 and each of the second polypeptides comprises the amino acid sequence of SEQ ID NO:45.

TABLE D
Peptide Chains of Example Bispecific Antibodies
C-1A10

| Heavy | Heavy | | EVQLVESGGGLVQPGGSLRLSCAASGFTFSTFGMHWVRQAPGKGLEWVS |
|---|---|---|---|
| component | chain of | | YITSGESPIYFTDTVKGRFTISRDNAKNSLYLQMNSLRAEDTAVYYCAR |
| (SEQ ID | 4F11E2 | | SSYYGNSMDYWGQGTLVTVSS (SEQ ID NO: 26) |
| NO: 40) | | | ASTKGPSVFPLAPSSKSTSGGTAALGCLVKDYFPEPVTVSWNSGALTSG |
| | | | VHTFPAVLQSSGLYSLSSVVTVPSSSLGTQTYICNVNHKPSNTKVDKKV |
| | | | EPKSCDKTHTCPPCPAPELLGGPSVFLFPPKPKDTLMISRTPEVTCVVV |
| | | | DVSHEDPEVKFNWYVDGVEVHNAKTKPREEQYASTYRVVSVLTVLHQDW |
| | | | LNGKEYKCKVSNKALPAPIEKTISKAKGQPREPQVYTLPPSREEMTKNQ |
| | | | VSLTCLVKGFYPSDIAVEWESNGQPENNYKTTPPVLDSDGSFFLYSKLT |
| | | | VDKSRWQQGNVFSCSVMHEALHNHYTQKSLSLSPGK (SEQ ID |
| | | | NO: 32) |
| | Linker | | GGGGSGGGGSGGGGS (SEQ ID NO: 34) |
| | scFv of | VL | QSVLTQPPSASGTPGQRVTISCSGSSSNIGNNYVTWYQQLPGTAPKLLI |
| | 1A10 | | YADSHRPSGVPDRFSGSKSGTSASLAISGLRSEDEADYYCA |
| | | | TWDYSLSGYVFGCGTKLTVL (SEQ ID NO: 25) |
| | | Linker | GGGGSGGGGSGGGGSGGGGS (SEQ ID NO: 35) |
| | | VH | EVQLLESGGGLVQPGGSLRLSCAASGFTFSSYDMSWVRQAPGKCLEWVS |
| | | | WISYSGGSIYYADSVKGRFTISRDNSKNTLYLQMNSLRAEDTAVYYCAR |
| | | | QNAYSYPFTWGQGTLVTVSS (SEQ ID NO: 24) |

| | | |
|---|---|---|
| Light | Light | DIVMTQSPDSLAVSLGERATINCRSSQSLLNAGNRKNYLTWYQQKPGQP |
| component | chain of | PKLLIYWASTRESGVPDRFSGSGSGTDFTLTISSLQAEDVAVYYCQ |
| (SEQ ID | 4F11E2 | NAYSYPFTFGGGTKLEIK (SEQ ID NO: 27) |
| NO: 41) | | |
| | | RTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPV |
| | | TKSFNRGEC (SEQ ID NO: 33) |

D-1A10

| Heavy | Heavy | | QVQLVQSGAEVKKPGASVKVSCKASGYTFTTYPIEWVRQAPGQRLEWMG |
|---|---|---|---|
| component | chain of | | NFHPYNDDTKYNEKFKGRVTITRDTSASTAYMELSSLRSEDTAVYYCAR |
| (SEQ ID | 72C1B6A3 | | RAYGYPYAMDYWGQGTLVTVSS (SEQ ID NO: 28) |
| NO: 42) | | | ASTKGPSVFPLAPSSKSTSGGTAALGCLVKDYFPEPVTVSWNSGALTSG |
| | | | VHTFPAVLQSSGLYSLSSVVTVPSSSLGTQTYICNVNHKPSNTKVDKKV |
| | | | EPKSCDKTHTCPPCPAPELLGGPSVFLFPPKPKDTLMISRTPEVTCVVV |
| | | | DVSHEDPEVKFNWYVDGVEVHNAKTKPREEQYASTYRVVSVLTVLHQDW |
| | | | LNGKEYKCKVSNKALPAPIEKTISKAKGQPREPQVYTLPPSREEMTKNQ |
| | | | VSLTCLVKGFYPSDIAVEWESNGQPENNYKTTPPVLDSDGSFFLYSKLT |
| | | | VDKSRWQQGNVFSCSVMHEALHNHYTQKSLSLSPGK (SEQ ID |
| | | | NO: 32) |
| | Linker | | GGGGSGGGGSGGGGS (SEQ ID NO: 34) |
| | scFv of | VL | QSVLTQPPSASGTPGQRVTISCSGSSSNIGNNYVTWYQQLPGTAPKLLI |
| | 1A10 | | YADSHRPSGVPDRFSGSKSGTSASLAISGLRSEDEADYYCA |
| | | | TWDYSLSGYVFGCGTKLTVL (SEQ ID NO: 25) |
| | | Linker | GGGGSGGGGSGGGGSGGGGS (SEQ ID NO: 35) |
| | | VH | EVQLLESGGGLVQPGGSLRLSCAASGFTFSSYDMSWVRQAPGKCLEWVS |
| | | | WISYSGGSIYYADSVKGRFTISRDNSKNTLYLQMNSLRAEDTAVYYCAR |
| | | | DAQRNSMREFDYWGQGTLVTVSS (SEQ ID NO: 24) |

| | | |
|---|---|---|
| Light | Light | DIVMTQSPDSLAVSLGERATINCKSSQSLLNAGNQKNYLTWYQQKPGQP |
| component | chain of | PKLLIYRASSRESGVPDRFSGSGSGTDFTLTISSLQAEDVAVYYCQNDY |
| | 72C1B6A3 | IYPYTFGGGTKLEIK (SEQ ID NO: 29) |
| (SEQ ID | | RTVAAPSVFIFPPSDEQLKSGTASVVCLLNNFYPREAKVQWKVDNALQS |
| NO: 43) | | GNSQESVTEQDSKDSTYSLSSTLTLSKADYEKHKVYACEVTHQGLSSPV |
| | | TKSFNRGEC (SEQ ID NO: 33) |

E-1A10

| Heavy | Heavy | | QVQLVQSGAEVKKPGASVKVSCKASGYTFTGYIIQWVRQAPGQRLEWMG |
|---|---|---|---|
| component | chain of | | FINPYNDDTKYNEQFKGRVTITRDTSASTAYMELSSLRSEDTAVYYCAR |
| (SEQ ID | 120B7B2 | | AYFGNAFAYWGQGTLVTVSS (SEQ ID NO: 30) |
| NO: 44) | | | ASTKGPSVFPLAPSSKSTSGGTAALGCLVKDYFPEPVTVSWNSGALTSG |
| | | | VHTFPAVLQSSGLYSLSSVVTVPSSSLGTQTYICNVNHKPSNTKVDKKV |
| | | | EPKSCDKTHTCPPCPAPELLGGPSVFLFPPKPKDTLMISRTPEVTCVVV |
| | | | DVSHEDPEVKFNWYVDGVEVHNAKTKPREEQYASTYRVVSVLTVLHQDW |
| | | | LNGKEYKCKVSNKALPAPIEKTISKAKGQPREPQVYTLPPSREEMTKNQ |
| | | | VSLTCLVKGFYPSDIAVEWESNGQPENNYKTTPPVLDSDGSFFLYSKLT |
| | | | VDKSRWQQGNVFSCSVMHEALHNHYTQKSLSLSPGK (SEQ ID |
| | | | NO: 32) |
| | Linker | | GGGGSGGGGSGGGGS (SEQ ID NO: 34) |
| | scFv of | VL | QSVLTQPPSASGTPGQRVTISCSGSSSNIGNNYVTWYQQLPGTAPKLLI |
| | 1A10 | | YADSHRPSGVPDRFSGSKSGTSASLAISGLRSEDEADYYCATWDYSLSG |
| | | | YVFGCGTKLTVL (SEQ ID NO: 25) |
| | | Linker | GGGGSGGGGSGGGGSGGGGS (SEQ ID NO: 35) |
| | | VH | EVQLLESGGGLVQPGGSLRLSCAASGFTFSSYDMSWVRQAPGKCLEWVS |
| | | | WISYSGGSIYYADSVKGRFTISRDNSKNTLYLQMNSLRAEDTAVYYCAR |
| | | | DAQRNSMREFDYWGQGTLVTVSS (SEQ ID NO: 24) |

| | | |
|---|---|---|
| Light | Light | DIVMTQSPDSLAVSLGERATINCKSSQSLLNAGNQKNYLTWYQQKPGQP |
| component | chain of | PKLLIYWASTRESGVPDRFSGSGSGTDFTLTISSLQAEDVAVYYCQNAY |
| (SEQ ID | 120B7B2 | YFPFTFGGGTKLEIK (SEQ ID NO: 31) |
| NO: 45) | | RTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPV |
| | | TKSFNRGEC (SEQ ID NO: 33) |

TABLE D2
Integrated Sequences of Example Bispecific Antibodies
C-1A10

| | |
|---|---|
| Heavy | EVQLVESGGGLVQPGGSLRLSCAASGFTFSTFGMHWVRQAPGKGLEWVSYITSGESPIYFT |
| component | DTVKGRFTISRDNAKNSLYLQMNSLRAEDTAVYYCARSSYYGNSMDYWGQGTLVTVSSASTKG |
| (SEQ ID | PSVFPLAPSSKSTSGGTAALGCLVKDYFPEPVTVSWNSGALTSGVHTFPAVLQSSGLYSLSS |
| NO: 40) | VVTVPSSSLGTQTYICNVNHKPSNTKVDKKVEPKSCDKTHTCPPCPAPELLGGPSVFLFPPK |
| | PKDTLMISRTPEVTCVVVDVSHEDPEVKFNWYVDGVEVHNAKTKPREEQYASTYRVVSVLTV |
| | LHQDWLNGKEYKCKVSNKALPAPIEKTISKAKGQPREPQVYTLPPSREEMTKNQVSLTCLVK |
| | GFYPSDIAVEWESNGQPENNYKTTPPVLDSDGSFFLYSKLTVDKSRWQQGNVFSCSVMHEAL |
| | HNHYTQKSLSLSPGKGGGGSGGGGSGGGGSQSVLTQPPSASGTPGQRVTISCSGSSSNIGNN |
| | YVTWYQQLPGTAPKLLIYADSHRPSGVPDRFSGSKSGTSASLAISGLRSEDEADYYCATWDY |
| | SLSGYVFGCGTKLTVLGGGGSGGGGSGGGGSGGGGSEVQLLESGGGLVQPGGSLRLSCAASG |
| | FTFSSYDMSWVRQAPGKCLEWVSWISYSGGSIYYADSVKGRFTISRDNSKNTLYLQMNSLRA |
| | EDTAVYYCARDAQRNSMREFDYWGQGTLVTVSS |
| Light | DIVMTQSPDSLAVSLGERATINCRSSQSLLNAGNRKNYLTWYQQKPGQPPKLLIYW |
| component | ASTRESGVPDRFSGSGSGTDFTLTISSLQAEDVAVYYCQNAYSYPFTFGGGTKLEIKRTVAAPSVFIF |
| (SEQ ID | PPSDEQLKSGTASVVCLLNNFYPREAKVQWKVDNALQSGNSQESVTEQDSKDSTYSLSSTLT |
| NO: 41) | LSKADYEKHKVYACEVTHQGLSSPVTKSFNRGEC |

D-1A10

| | |
|---|---|
| Heavy | QVQLVQSGAEVKKPGASVKVSCKASGYTFTTYPIEWVRQAPGQRLEWMGNFHPY |
| component | NDDTKYNEKFKGRVTITRDTSASTAYMELSSLRSEDTAVYYCARRAYGYPYAMDYWGQGTLVTVSSASTK |
| (SEQ ID | GPSVFPLAPSSKSTSGGTAALGCLVKDYFPEPVTVSWNSGALTSGVHTFPAVLQSSGLYSLS |
| NO: 42) | SVVTVPSSSLGTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQYASTYRVVSVLT |
| | VLHQDWLNGKEYKCKVSNKALPAPIEKTISKAKGQPREPQVYTLPPSREEMTKNQVSLTCLV |
| | KGFYPSDIAVEWESNGQPENNYKTTPPVLDSDGSFFLYSKLTVDKSRWQQGNVFSCSVMHEA |
| | LHNHYTQKSLSLSPGKGGGGSGGGGSGGGGSQSVLTQPPSASGTPGQRVTISCSGSSSNIGNNYVT |
| | WYQQLPGTAPKLLIYADSHRPSGVPDRFSGSKSGTSASLAISGLRSEDEADYYCATWDYSL |
| | SGYVFGCGTKLTVLGGGGSGGGGSGGGGSGGGGSEVQLLESGGGLVQPGGSLRLSCAAS |
| | GFTFSSYDMSWVRQAPGKCLEWVSWISYSGGSIYYADSVKGRFTISRDNSKNTLYLQMNSLR |
| | AEDTAVYYCARDAQRNSMREFDYWGQGTLVTVSS |
| Light | DIVMTQSPDSLAVSLGERATINCKSSQSLLNAGNQKNYLTWYQQKPGQPPKLLIYR |
| component | ASSRESGVPDRFSGSGSGTDFTLTISSLQAEDVAVYYCQNDYIYPYTFGGGTKLEIKRTVAAPSVFIF |
| (SEQ ID | PPSDEQLKSGTASVVCLLNNFYPREAKVQWKVDNALQSGNSQESVTEQDSKDSTYSLSSTLT |
| NO: 43) | LSKADYEKHKVYACEVTHQGLSSPVTKSFNRGEC |

E-1A10

| | |
|---|---|
| Heavy | QVQLVQSGAEVKKPGASVKVSCKASGYTFTGYIIQWVRQAPGQRLEWMGFINPYN |
| component | DDTKYNEQFKGRVTITRDTSASTAYMELSSLRSEDTAVYYCARAYFGNAFAYWGQGTLVTVSSASTKGP |
| (SEQ ID | SVFPLAPSSKSTSGGTAALGCLVKDYFPEPVTVSWNSGALTSGVHTFPAVLQSSGLYSLSSV |
| NO: 44) | VTVPSSSLGTQTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQYASTYRVVSVLTVL |
| | HQDWLNGKEYKCKVSNKALPAPIEKTISKAKGQPREPQVYTLPPSREEMTKNQVSLTCLVKG |
| | FYPSDIAVEWESNGQPENNYKTTPPVLDSDGSFFLYSKLTVDKSRWQQGNVFSCSVMHEALH |
| | NHYTQKSLSLSPGKGGGGSGGGGSGGGGSQSVLTQPPSASGTPGQRVTISCSGSSSNIGNN |
| | YVTWYQQLPGTAPKLLIYADSHRPSGVPDRFSGSKSGTSASLAISGLRSEDEADYYCATWDYSLSGYV |
| | FGCGTKLTVLGGGGSGGGGSGGGGSGGGGSEVQLLESGGGLVQPGGSLRLSCAASGF |
| | TFSSYDMSWVRQAPGKCLEWVSWISYSGGSIYYADSVKGRFTISRDNSKNTLYLQMNSLRAE |
| | DTAVYYCARDAQRNSMREFDYWGQGTLVTVSS |
| Light | DIVMTQSPDSLAVSLGERATINCKSSQSLLNAGNQKNYLTWYQQKPGQPPKLLIYW |
| component | ASTRESGVPDRFSGSGSGTDFTLTISSLQAEDVAVYYCQNAYFPFTFGGGTKLEIKRTVAAPSVFIF |
| (SEQ ID | PPSDEQLKSGTASVVCLLNNFYPREAKVQWKVDNALQSGNSQESVTEQDSKDSTYSLSSTLT |
| NO: 45) | LSKADYEKHKVYACEVTHQGLSSPVTKSFNRGEC |

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

In certain embodiments, the antibody comprises an amino acid sequence or one or more moieties not normally associated with an antibody. Exemplary modifications are described in more detail below. For example, an antibody of the disclosure may comprise a flexible linker sequence, or may be modified to add a functional moiety (e.g., PEG, a drug, a toxin, or a label).

Antibodies, variants, or derivatives thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

In some embodiments, the antibodies may be conjugated to therapeutic agents, prodrugs, peptides, proteins, enzymes, viruses, lipids, biological response modifiers, pharmaceutical agents, or PEG.

The antibodies may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

The antibodies can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

The antibodies can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA). Techniques for conjugating various moieties to an antibody are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al., (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. (52:119-58 (1982)).

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Treatment Methods

As described herein, the antibodies, variants or derivatives of the present disclosure may be used in certain treatment and diagnostic methods.

The present disclosure is further directed to antibody-based therapies which involve administering the antibodies of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

In some embodiments, provided are methods for treating a cancer in a patient in need thereof. The method, in one embodiment, entails administering to the patient an effective amount of an antibody of the present disclosure. In some embodiments, at least one of the cancer cells (e.g., stromal cells) in the patient over-express claudin 18.2.

Cellular therapies, such as chimeric antigen receptor (CAR) T-cell therapies, are also provided in the present disclosure. A suitable cell can be used, that is put in contact with an antibody of the present disclosure (or alternatively engineered to express an antibody of the present disclosure). Upon such contact or engineering, the cell can then be introduced to a cancer patient in need of a treatment. The cancer patient may have a cancer of any of the types as disclosed herein. The cell (e.g., T cell) can be, for instance, a tumor-infiltrating T lymphocyte, a CD4+ T cell, a CD8+ T cell, or the combination thereof, without limitation.

In some embodiments, the cell was isolated from the cancer patient him- or her-self. In some embodiments, the cell was provided by a donor or from a cell bank. When the cell is isolated from the cancer patient, undesired immune reactions can be minimized.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer. In some embodiments, the cancer is one or more of gastric, pancreatic, esophageal, ovarian, and lung cancers.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibodies, variants or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antigen-binding polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The amount of the antibodies of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antigen-binding polypeptides of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-□.

In additional embodiments, the compositions of the disclosure are administered in combination with other therapeutic or prophylactic regimens, such as, for example, radiation therapy.

### Compositions

The present disclosure also provides pharmaceutical compositions. Such compositions comprise an effective amount of an antibody, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1: Generation of Anti-CLDN18.2/4-1BB Bispecific Antibodies

Three previously identified anti-CLDN18.2 antibodies, 4F11E2, 72C1B6A3 and 120B7B2, and an anti-4-1BB antibody, 1A10 (sequences are shown in the tables below), were selected to generate anti-CLDN18.2-4-1BB bispecific antibodies in a full-length IgG X scFv form (structure illustrated in FIG. 1A). The anti-Claudin 18.2 portion was placed in full IgG part, while the anti-4-1BB was a scFv placed at the C-terminal side of the Fc fragment. The bispecific antibodies included an IgG1 backbone with a N297A mutation to disable Fc function.

TABLE 1
Antibody Sequences
C-1A10

| Heavy | Heavy chain of | | EVQLVESGGGLVQPGGSLRLSCAASGFTFSTFGMHWVRQAPGKGLEWVS |
|---|---|---|---|
| component | 4F11E2 | | YITSGESPIYFTDTVKGRFTISRDNAKNSLYLQMNSLRAEDTAVYYCAR |
| | | | SSYYGNSMDYWGQGTLVTVSS (SEQ ID NO: 26) |
| | | | ASTKGPSVFPLAPSSKSTSGGTAALGCLVKDYFPEPVTVSWNSGALTSG |
| | | | VHTFPAVLQSSGLYSLSSVVTVPSSSLGTQTYICNVNHKPSNTKVDKKV |
| | | | EPKSCDKTHTCPPCPAPELLGGPSVFLFPPKPKDTLMISRTPEVTCVVV |
| | | | DVSHEDPEVKFNWYVDGVEVHNAKTKPREEQYASTYRVVSVLTVLHQDW |
| | | | LNGKEYKCKVSNKALPAPIEKTISKAKGQPREPQVYTLPPSREEMTKNQ |
| | | | VSLTCLVKGFYPSDIAVEWESNGQPENNYKTTPPVLDSDGSFFLYSKLT |
| | | | VDKSRWQQGNVFSCSVMHEALHNHYTQKSLSLSPGK (SEQ ID |
| | | | NO: 32) |
| | Linker | | GGGGSGGGGSGGGGS (SEQ ID NO: 34) |
| | scFv of | VL | QSVLTQPPSASGTPGQRVTISCSGSSSNIGNNYVTWYQQLPGTAPKLLI |
| | 1A10 | | YADSHRPSGVPDRFSGSKSGTSASLAISGLRSEDEADYYCA |
| | | | TWDYSLSGYVFGCGTKLTVL (SEQ ID NO: 25) |
| | | Linker | GGGGSGGGGSGGGGSGGGGS(SEQ ID NO: 35) |
| | | VH | EVQLLESGGGLVQPGGSLRLSCAASGFTESSYDMSWVRQAPGKCLEWVS |
| | | | WISYSGGSIYYADSVKGRFTISRDNSKNTLYLQMNSLRAEDTAVYYCAR |
| | | | DAQRNSMREFDYWGQGTLVTVSS (SEQ ID NO: 24) |

| | | |
|---|---|---|
| Light | Light chain of | DIVMTQSPDSLAVSLGERATINCRSSQSLLNAGNRKNYLTWYQQKPGQP |
| component | 4F11E2 | PKLLIYWASTRESGVPDRFSGSGSGTDFTLTISSLQAEDVAVYYC |
| | | QNAYSYPFTFGGGTKLEIK (SEQ ID NO: 27) |
| | | RTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPV |
| | | TKSFNRGEC (SEQ ID NO: 33) |

D-1A10

| Heavy | Heavy chain of | | QVQLVQSGAEVKKPGASVKVSCKASGYTFTTYPIEWVRQAPGQRLEWMG |
|---|---|---|---|
| component | 72C1B6A3 | | NFHPYNDDTKYNEKFKGRVTITRDTSASTAYMELSSLRSEDTAVYYCAR |
| | | | RAYGYPYAMDYWGQGTLVTVSS (SEQ ID NO: 28) |
| | | | ASTKGPSVFPLAPSSKSTSGGTAALGCLVKDYFPEPVTVSWNSGALTSG |
| | | | VHTFPAVLQSSGLYSLSSVVTVPSSSLGTQTYICNVNHKPSNTKVDKKV |
| | | | EPKSCDKTHTCPPCPAPELLGGPSVFLFPPKPKDTLMISRTPEVTCVVV |
| | | | DVSHEDPEVKFNWYVDGVEVHNAKTKPREEQYARVVSVLTVLHQDW |
| | | | LNGKEYKCKVSNKALPAPIEKTISKAKGQPREPQVYTLPPSREEMTKNQ |
| | | | VSLTCLVKGFYPSDIAVEWESNGQPENNYKTTPPVLDSDGSFFLYSKLT |
| | | | VDKSRWQQGNVFSCSVMHEALHNHYTQKSLSLSPGK (SEQ ID |
| | | | NO: 32) |
| | Linker | | GGGGSGGGGSGGGGS (SEQ ID NO: 34) |
| | scFv of | VL | QSVLTQPPSASGTPGQRVTISCSGSSSNIGNNYVTWYQQLPGTAPKLLI |
| | 1A10 | | YADSHRPSGVPDRFSGSKSGTSASLAISGLRSEDEADYYCA |
| | | | TWDYSLSGYVFGCGTKLTVL (SEQ ID NO: 25) |
| | | Linker | GGGGSGGGGSGGGGSGGGGS (SEQ ID NO: 35) |
| | | VH | EVQLLESGGGLVQPGGSLRLSCAASGFTFSSYDMSWVRQAPGKCLEWVS |
| | | | WISYSGGSIYYADSVKGRFTISRDNSKNTLYLQMNSLRAEDTAVYYCAR |
| | | | DAQRNSMREFDYWGQGTLVTVSS (SEQ ID NO: 24) |

| | | |
|---|---|---|
| Light | Light chain of | DIVMTQSPDSLAVSLGERATINCKSSQSLLNAGNQKNYLTWYQQKPGQP |
| component | 72C1B6A3 | PKLLIYRASSRESGVPDRFSGSGSGTDFTLTISSLQAEDVAVYYCQNDY |
| | | IYPYTFGGGTKLEIK (SEQ ID NO: 29) |
| | | RTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPV |
| | | TKSFNRGEC (SEQ ID NO: 33) |

E-1A10

| Heavy | Heavy chain of | | QVQLVQSGAEVKKPGASVKVSCKASGYTFTGYIIQWVRQAPGQRLEWMG |
|---|---|---|---|
| component | 120B7B2 | | FINPYNDDTKYNEQFKGRVTITRDTSASTAYMELSSLRSEDTAVYYCAR |
| | | | AYFGNAFAYWGQGTLVTVSS (SEQ ID NO: 30) |
| | | | ASTKGPSVFPLAPSSKSTSGGTAALGCLVKDYFPEPVTVSWNSGALTSG |
| | | | VHTFPAVLQSSGLYSLSSVVTVPSSSLGTQTYICNVNHKPSNTKVDKKV |
| | | | EPKSCDKTHTCPPCPAPELLGGPSVFLFPPKPKDTLMISRTPEVTCVVV |
| | | | DVSHEDPEVKFNWYVDGVEVHNAKTKPREEQYASTYRVVSVLTVLHQDW |
| | | | LNGKEYKCKVSNKALPAPIEKTISKAKGQPREPQVYTLPPSREEMTKNQ |
| | | | VSLTCLVKGFYPSDIAVEWESNGQPENNYKTTPPVLDSDGSFFLYSKLT |
| | | | VDKSRWQQGNVFSCSVMHEALHNHYTQKSLSLSPGK (SEQ ID |
| | | | NO: 32) |
| | Linker | | GGGGSGGGGSGGGGS(SEQ ID NO: 34) |
| | scFv of | VL | QSVLTQPPSASGTPGQRVTISCSGSSSNIGNNYVTWYQQLPGTAPKLLI |
| | 1A10 | | YADSHRPSGVPDRFSGSKSGTSASLAISGLRSEDEADYYCA |
| | | | TWDYSLSGYVFGCGTKLTVL (SEQ ID NO: 25) |
| | | Linker | GGGGSGGGGSGGGGSGGGGS(SEQ ID NO: 35) |
| | | VH | EVQLLESGGGLVQPGGSLRLSCAASGFTFSSYDMSWVRQAPGKCLEWVS |
| | | | WISYSGGSIYYADSVKGRFTISRDNSKNTLYLQMNSLRAEDTAVYYCAR |
| | | | DAQRNSMREFDYWGQGTLVTVSS (SEQ ID NO: 24) |

| | | |
|---|---|---|
| Light | Light chain of | DIVMTQSPDSLAVSLGERATINCKSSQSLLNAGNQKNYLTWYQQKPGQP |
| component | 120B7B2 | PKLLIYWASTRESGVPDRFSGSGSGTDFTLTISSLQAEDVAVYYCQNAY |
| | | YFPFTFGGGTKLEIK (SEQ ID NO: 31) |
| | | RTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPV |
| | | TKSFNRGEC (SEQ ID NO: 33) |

The 4F11E2 and 1A10 sequences were also used to generate bispecific antibodies of two different formats, as illustrated in FIG. 1B and FIG. 1C, respectively. In the format of FIG. 1B, the anti-Claudin 18.2 portion also took a Fab format, while the anti-4-1BB portion was present as a scFab fragment inserted between the anti-Claudin 18.2 Fab and the Fc fragments. The IgG1 (N297A) was also used here.

TABLE 2
Antibody of Format of FIG. 1B
Fab-scFab-Fc

| Heavy | Heavy | | EVQLVESGGGLVQPGGSLRLSCAASGFTFSTFGMHWVRQAPGKGLEWV |
|---|---|---|---|
| component | chain of | | SYITSGESPIYFTDTVKGRFTISRDNAKNSLYLQMNSLRAEDTAVYYC |
| (SEQ ID | 4F11E2 | | ARSSYYGNSMDYWGQGTLVTVSS(SEQ ID NO: 26) |
| NO: 54) | | | ASTKGPSVFPLAPSSKSTSGGTAALGCLVKDYFPEPVTVSWNSGALTS |
| | | | GVHTFPAVLQSSGLYSLSSVVTVPSSSLGTQTYICNVNHKPSNTKVDK |
| | | | KVEPKSC (SEQ ID NO: 36) |
| | Linker | | GGGGSGGGGSGGGGSGGGGS (SEQ ID NO: 35) |
| | scFab | Light | QSVLTQPPSASGTPGQRVTISCSGSSSNIGNNYVTWYQQLPGTAPKLL |
| | of | chain | IYADSHRPSGVPDRFSGSKSGTSASLAISGLRSEDEADYYCATWDYSL |
| | 1A10 | of | SGYVFGGGTKLTVL (SEQ ID NO: 53) |
| | | 1A10 | GQPKANPTVTLFPPSSEELQANKATLVCLISDFYPGAVTVAWKADGSP |
| | | | VKAGVETTKPSKQSNNKYAASSYLSLTPEQWKSHRSYSCQVTHEGSTV |
| | | | EKTVAPTECS (SEQ ID NO: 37) |
| | | Linker | GSGSGSGSGSGSGSGSGSGGGGSGGGGSGGGGSGGGGSGGGGSGGGGS |
| | | | GSGSGSGSGSGSGSGS (SEQ ID NO: 38) |
| | | Heavy | EVQLLESGGGLVQPGGSLRLSCAASGFTFSSYDMSWVRQAPGKGLEWV |
| | | chain | SWISYSGGSIYYADSVKGRFTISRDNSKNTLYLQMNSLRAEDTAVYYC |
| | | of | ARDAQRNSMREFDYWGQGTLVTVSS (SEQ ID NO: 47) |
| | | 1A10 | ASTKGPSVFPLAPSSKSTSGGTAALGCLVKDYFPEPVTVSWNSGALTS |
| | | | GVHTFPAVLQSSGLYSLSSVVTVPSSSLGTQTYICNVNHKPSNTKVDK |
| | | | KVEPKSCDKTHTCPPCPAPELLGGPSVFLFPPKPKDTLMISRTPEVTC |
| | | | VVVDVSHEDPEVKFNWYVDGVEVHNAKTKPREEQYASTYRVVSVLTVL |
| | | | HQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFF |
| | | | LYSKLTVDKSRWQQGNVFSCSVMHEALHNHYTQKSLSLSPGK (SEQ |
| | | | ID NO: 32) |

| | | |
|---|---|---|
| Light | Light | DIVMTQSPDSLAVSLGERATINCRSSQSLLNAGNRKNYLTWYQQKPGQ |
| component | chain of | PPKLLIYWASTRESGVPDRFSGSGSGTDFTLTISSLQAEDVAVYYCQN |
| (SEQ ID | 4F11E2 | AYSYPFTFGGGTKLEIK (SEQ ID NO: 27) |
| NO: 41) | | RTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSS |
| | | PVTKSFNRGEC (SEQ ID NO: 33) |

In the format of FIG. 1C, the anti-Claudin 18.2 portion was present in full IgG1, while the anti-4-1BB portion was a scFab fragment placed at the C-terminal side of the Fc.

TABLE 3
Antibody of Format of FIG. 1C
IgG-scFab

| Heavy | Heavy | | EVQLVESGGGLVQPGGSLRLSCAASGFTFSTFGMHWVRQAPGKGLE |
|---|---|---|---|
| component | chain of | | WVSYITSGESPIYFTDTVKGRFTISRDNAKNSLYLQMNSLRAEDTA |
| (SEQ ID | 4F11E2 | | VYYCARSSYYGNSMDYWGQGTLVTVSS(SEQ ID NO: 26) |
| NO: 55) | | | ASTKGPSVFPLAPSSKSTSGGTAALGCLVKDYFPEPVTVSWNSGAL |
| | | | TSGVHTFPAVLQSSGLYSLSSVVTVPSSSLGTQTYICNVNHKPSNT |
| | | | KVDKKVEPKSCDKTHTCPPCPAPELLGGPSVFLFPPKPKDTLMISR |
| | | | TPEVTCVVVDVSHEDPEVKFNWYVDGVEVHNAKTKPREEQYASTYR |
| | | | VVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQK |
| | | | SLSLSPGK (SEQ ID NO: 32) |
| | Linker | | GGGGSGGGGSGGGGS (SEQ ID NO: 34) |
| | scFab | Light | QSVLTQPPSASGTPGQRVTISCSGSSSNIGNNYVTWYQQLPGTAPK |
| | of | chain | LLIYADSHRPSGVPDRFSGSKSGTSASLAISGLRSEDEADYYCATW |
| | 1A10 | of | DYSLGYVFGGGTKLTVL (SEQ ID NO: 53) |
| | | 1A10 | GQPKANPTVTLFPPSSEELQANKATLVCLISDFYPGAVTVAWKADG |
| | | | SPVKAGVETTKPSKQSNNKYAASSYLSLTPEQWKSHRSYSCQVTHE |
| | | | GSTVEKTVAPTECS (SEQ ID NO: 37) |
| | | Linker | GSGSGSGSGSGSGSGSGSGGGGSGGGGSGGGGSGGGGSGGGGSGGG |
| | | | GSGSGSGSGSGSGSGSGS (SEQ ID NO: 38) |
| | | Heavy | EVQLLESGGGLVQPGGSLRLSCAASGFTFSSYDMSWVRQAPGKGLE |
| | | chain | WVSWISYSGGSIYYADSVKGRFTISRDNSKNTLYLQMNSLRAEDTA |
| | | of | VYYCARDAQRNSMREFDYWGQGTLVTVSS(SEQ ID NO: 47) |
| | | 1A10 | ASTKGPSVFPLAPSSKSTSGGTAALGCLVKDYFPEPVTVSWNSGAL |
| | | | TSGVHTFPAVLQSSGLYSLSSVVTVPSSSLGTQTYICNVNHKPSNT |
| | | | KVDKKVEPKSC (SEQ ID NO: 36) |

| | | |
|---|---|---|
| Light | Light | DIVMTQSPDSLAVSLGERATINCRSSQSLLNAGNRKNYLTWYQQKP |
| component | chain of | GQPPKLLIYWASTRESGVPDRFSGSGSGTDFTLTISSLQAEDVAVY |
| (SEQ ID | 4F11E2 | YCQNAYSYPFTFGGGTKLEIK (SEQ ID NO: 27) |
| NO: 41) | | RTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQ |
| | | GLSSPVTKSFNRGEC (SEQ ID NO: 33) |

The bispecific antibodies of the formats in FIGS. 1B and 1C were outperformed by those of format in FIG. 1A in terms of 4-1BB binding, in preliminary testing. Thus, only those of format FIG. 1A underwent additional testing as shown below. Further, E-1A10 was slightly less potent than C-1A10 and D-1A10 and was thus also not included in further studies.

### Example 2. Antigen Binding of the Anti-CLDN18.2/4-1BB Bispecific Antibodies

This example evaluated the binding activities of the bispecific antibodies of Example 1 to CLDN18.2 and 4-1BB, using protein-based and cell-based assays.

### 2.1 ELISA Binding to 4-1BB

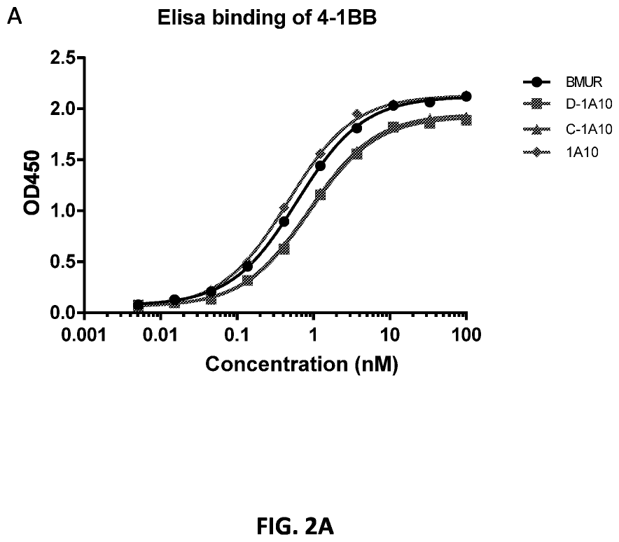

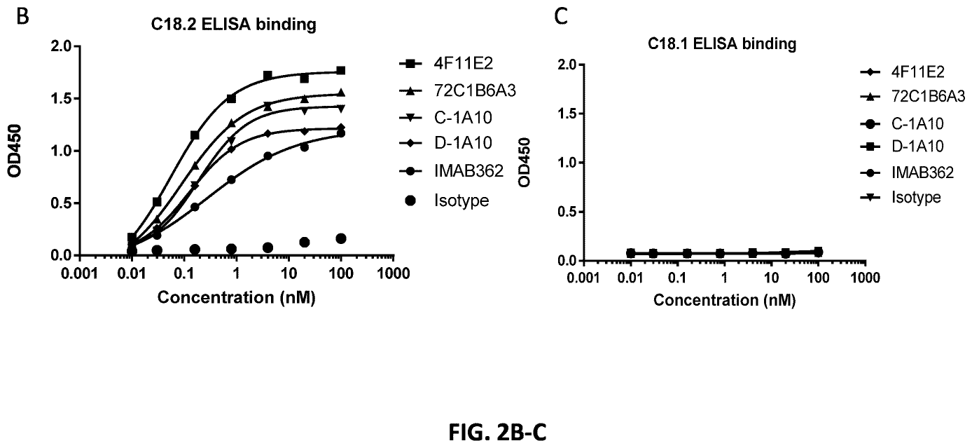

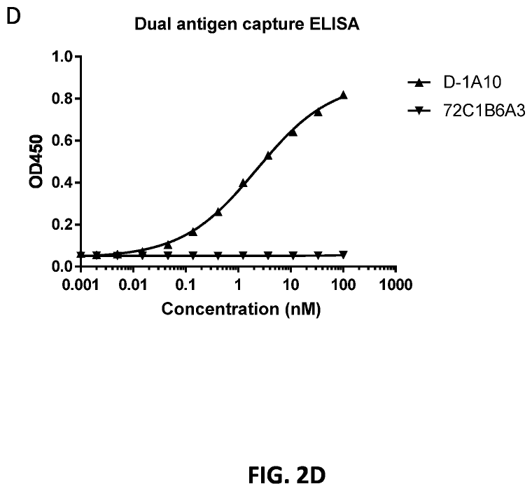

Briefly, microtiter plates were coated with 100 μl human 4-1BB-His protein at 0.5 μg/ml diluted in PBS at 4° C. overnight, then blocked with 100 μl/well of 1% BSA. Three-fold dilution of antibodies starting from 100 nM were added to each well and incubated for 2 hours at RT. The plates were washed with PBS/Tween and then incubated with goat-anti-human IgG antibody conjugated with Horse Radish Peroxidase (HRP) for 30 minutes at RT. After washing, the plates were developed with TMB substrate and analyzed by plate reader at OD450 nm. As shown in FIG. 2A, the anti-CLDN18.2-4-1BB antibodies (C-1A10 and D-1A10) showed comparable binding as anti-4-1BB monoclonal antibodies (1A10, and urelumab (BMUR)).

### 2.2 ELISA Binding to CLDN18.2

To text CLDN18.2 binding, the CLDN18.2 protein was expressed in a Virus Like Particle (VLP) to mimic the nature conformation. Similarly, microtiter plates were coated with 100 μl CLDN18.2 VLP at 3 μg/ml diluted in PBS at 4° C. overnight, then blocked with 300 μl/well of 3% BSA. Three-fold dilution of antibodies starting from 100 nM were added to each well and incubated for 2 hours at 37° C. The plates were washed with PBS/Tween and then incubated with goat-anti-human IgG antibody conjugated with Horse Radish Peroxidase (HRP) for 30 minutes at RT. After washing, the plates were developed with TMB substrate and analyzed by plate reader at OD450 nm. As shown in FIG. 2B, the anti-CLDN18.2-4-1BB antibodies (C-1A10 and D-1A10) showed stronger binding than the anti-CLDN18.2 monoclonal antibody (I-MAB362). Meanwhile, in the same experiment setting, the binding to CLDN18.1 protein was tested as well. As shown in FIG. 2C, no antibody showed cross-reactivity to CLDN18.1.

### 2.3 ELISA Binding to Dual Antigen

To further demonstrate that the anti-CLDN18.2-4-1BB antibodies can bind to CLDN18.2 and 4-1BB simultaneously, a DACE (Dual antigen captured ELISA) test was performed. Briefly, microtiter plate was coated with 100 μl CLDN18.2 VLP at 3 μg/ml diluted in PBS at 4° C. overnight. Then, three-fold dilution of antibodies starting from 100 nM were added to each well and incubated for 2 hours at 37° C. After washing, the plate was then incubated with 100 μl human 4-1BB-biotin protein at a concentration of lug/ml for 1 hour at 37° C. and followed with Streptavidin HRP for another 30 minutes at room temperature. After washing, the plates were developed with TMB substrate and analyzed by plate reader at OD 450 nm. As shown in FIG. 2D, D-1A10 can bind to hCLDN18.2 and 4-1BB at the same time. On the contrast, CLDN18.2 mAb 72C1B6A3 showed no signal in this assay.

### 2.4 Cell-Based Binding to 4-1BB

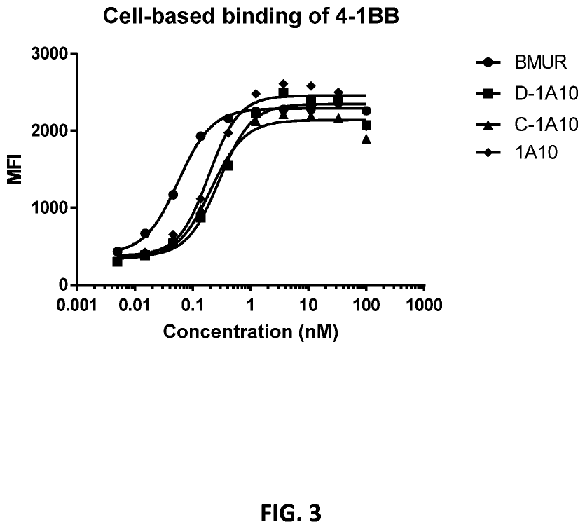

To evaluate the antigen binding property, the anti-CLDN18.2-4-1BB antibodies were analyzed for their binding to HEK293 expressed 4-1BB by FACS. A total number of 1×10⁵ HEK293-4-1BB cells in each well were incubated with serial diluted antibodies for 30 minutes at 4° C. in FACS buffer (2% FBS in PBS). After wash by FACS buffer, PE conjugated-anti-human IgG antibody was added to each well and incubated at 4° C. for 30 minutes. After wash, MFI of PE was evaluated by FACS Caliber. As shown in FIG. 3, the anti-CLDN18.2-4-1BB antibodies (C-1A10 and D-1A10) tested showed concentration-dependent binding abilities to 4-1BB, comparable with the monoclonal antibodies (1A10 and BMUR).

### 2.5 Cell-Based Binding to CLDN18.2

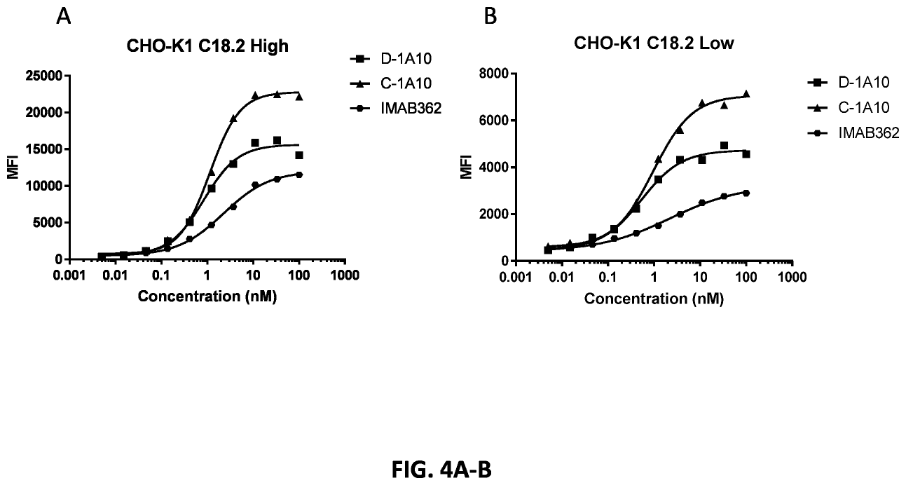

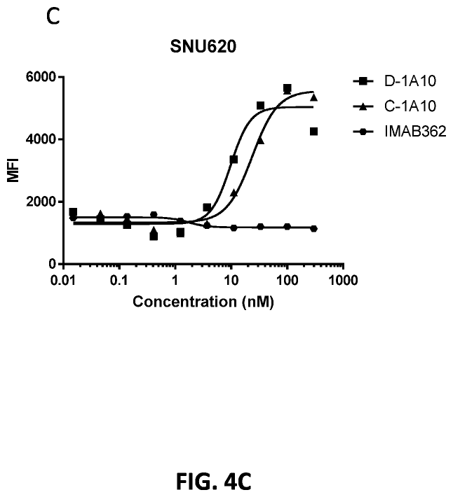

To evaluate the binding capability towards CLDN18.2, a CHO-K1 cell line which stably expressed human CLDN18.2 was made. CHO-C18.2 cell line was then sorted for high expressers (CHO-K1 C18.2 High) and low expressers (CHO-K1 C18.2 Low) using flow cytometry. CHO-K1-C18.2 cells were incubated with serial diluted antibodies for 30 minutes at 4° C. in FACS buffer. After wash by FACS buffer, PE conjugated-anti-human IgG antibody was added and incubated at 4° C. for 30 minutes. MFI of PE was evaluated by FACS. As shown in FIGS. 4A and 4B, anti-CLDN18.2-4-1BB antibodies bound to CLDN18.2-expressed cells in a concentration-dependent manner; meanwhile, both of the bispecific antibodies showed stronger binding than IMAB362, a reference monoclonal anti-CLDN18.2 antibody currently in clinical trials.

SNU620 is a gastric carcinoma cell line with endogenous CLDN18.2 expression. As shown in FIG. 4C, the anti-CLDN18.2-4-1BB bispecific antibodies could bind to SNU620 as well.

### Example 3. Functional Activity of Anti-CLDN18.2/4-1BB Bispecific Antibodies

### 3.1 Cell-Line Based Functional Characterization of CLDN18.2-4-1BB Bispecific Antibody

To test the ability of the bispecific antibodies to promote 4-1BB signal, a commercial 4-1BB assay was used. In this assay, GloResponse™ NFKB-luc2/4-1BB Jurkat cell line (Promega, cat #CS196004) was used as effector cells and CHO-K1-expressing or not expressing CLDN18.2 or SNU620 as target cells. GloResponse™ NFKB-luc2/4-1BB Jurkat cell line is genetically modified to stably express 4-1BB and luciferase downstream of a response element. Luciferase expression is induced upon antibody binding to the 4-1BB receptor. In brief, effector cells at a density of 2.5×10⁴ cells per well were mixed with 2.5×10⁴ target cells in a white 96-well plate. Antibodies 6-fold serially diluted were added to a white 96-well assay plate, at a final concentration ranging from 16.7 nM to 0.28 fm. After 6 hours' incubation at 37° C., luminescence was obtained by adding the substrate of luciferase and measured by a microplate reader (PHERAstar). Four-parameter logistic curve analysis was performed with GraphPad software.

As shown in FIG. 5, the BMUR monoclonal antibody can dose-dependent boost the 41BB singling, while the 1A10 monoclonal antibody had no agonist activity in the same experimental settings. The activity of anti-CLDN18.2-4-1BB bispecific antibodies D-1A10 and C-1A10 was dependent on the expression of CLDN18.2.

### 3.2 Activity of the Bispecific Antibodies to Promote Human Peripheral Blood Mononuclear Cell (PBMC) Immune Response

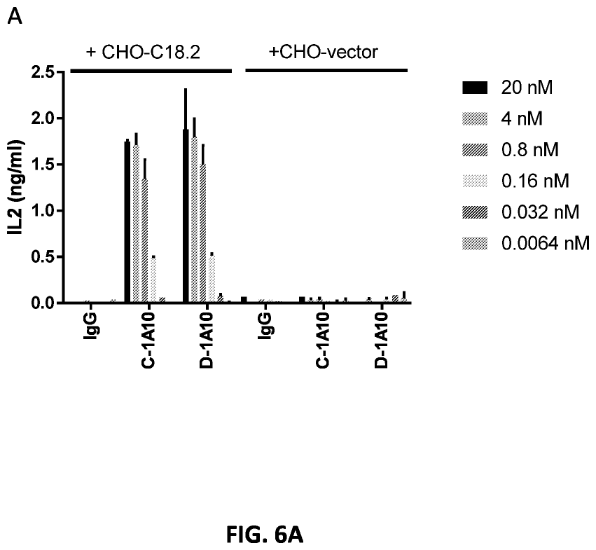

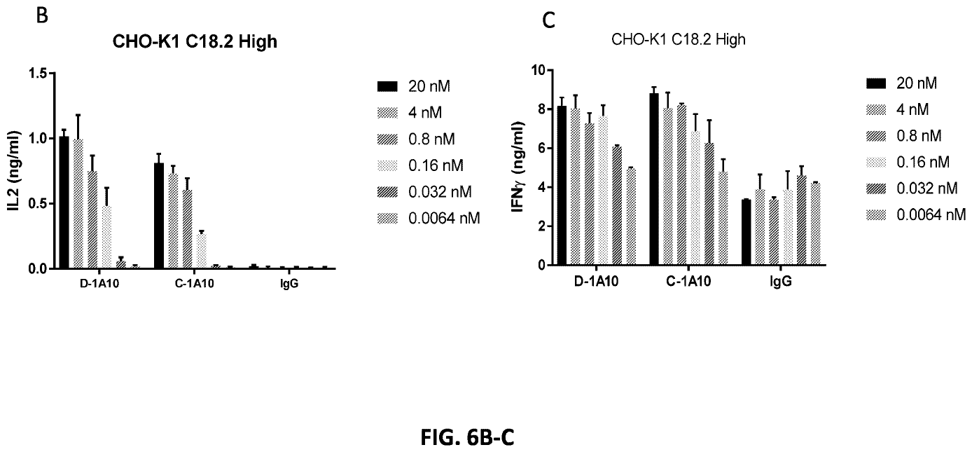

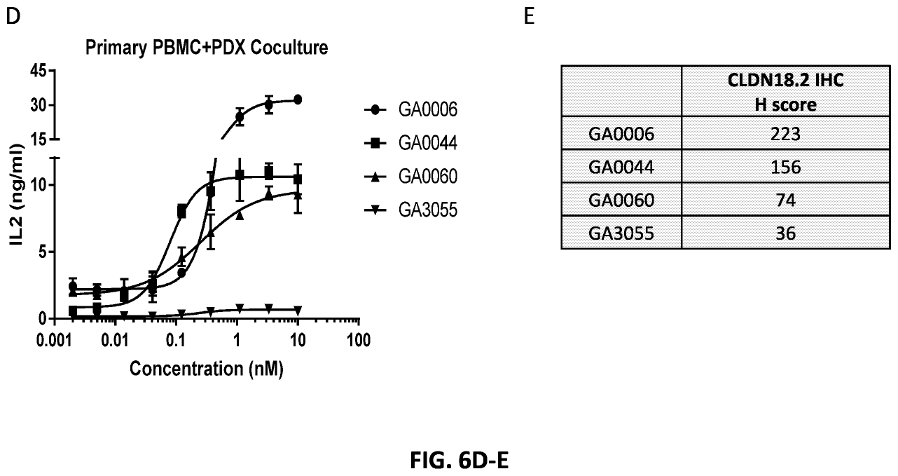

To test the ability of bispecific antibodies to stimulated human PBMCs response, cytokine production assay was used. Human PBMCs stimulated with 0.5 0 g/ml human anti-CD3 antibody were used as the effector cells. CHO-K1 express CLDN18.2 was used as the target cells. Human PBMCs (1×10⁵) were co-cultured with CHO-K1-CLDN18.2 or control CHO-K1 cells (2.5×10⁴) in the presence of human anti-CD3 antibody. Serially diluted bispecific antibodies were added to the mixed culture at a final concentration starting from 20 nM. 48 hours later, the level of IL2 and IFN-γ in the culture medium was measured using IL-2 (human) LANCE Ultra TR-FRET Detection Kit and IFN-γ (human) LANCE Ultra TR-FRET Detection Kit (PerkinElmer). As shown in FIG. 6, only bispecific antibodies can activate PBMC response in the presence of CLDN18.2 expressing cells.

To further demonstrate that the activity of bispecific antibodies was correlated with CLDN18.2 level, human PBMC from one donor were cocultured with different Gastric Adenocarcinoma (GA) PDX derived cells, which was proved to express different level of C18.2. Serially diluted bispecific antibodies were added to the mixed culture at a final concentration starting from 20 nM. After 48 hours, the level of IL2 in the culture medium was measured using IL-2 (human) LANCE Ultra TR-FRET Detection Kit (PerkinElmer). As shown in FIGS. 6D and 6E, the activity of bispecific antibody D-1A10 correlated with CLDN18.2 level. D-1A10 effectively activated T cells even in CLDN18.2 low-to middle-expressed tumors.

### 3.3 Activity of the Bispecific Antibodies to Promote Human CD8+ T Cell Response

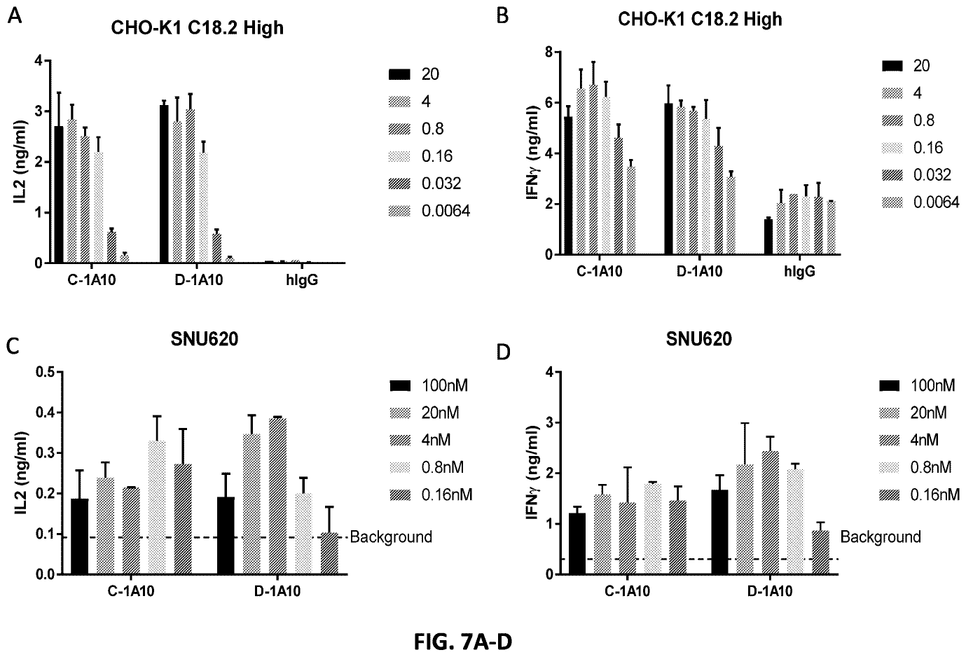

To further test the ability of bispecific antibodies in activating human CD8+ T cell, human CD8+ T cells isolated from PBMCs were used as the effector cells. CHO-K1 expressed CLDN18.2 or SNU620 was used as the target cells. Isolated CD8+ T cells (7.5×10⁴) were co-cultured with target cells (2.5×10⁴) in the presence of human anti-CD3 antibody. Serially diluted bispecific antibodies were added to the mixed culture at a final concentration starting from 20 nM. The level of IL2 and IFN-γ in the culture medium was measured 48 hours after coculture, using IL-2 (human) LANCE Ultra TR-FRET Detection Kit and IFN-γ (human) LANCE Ultra TR-FRET Detection Kit (PerkinElmer). As shown in FIG. 7, bispecific antibodies C-1A10 and D-1A10 can both increase the production of IL2 and IFN-γ of activated CD8+ T cells in the presence of C18.2 overexpressing CHO-K1 (FIG. 7A-7B) or SNU620 which endogenously express C18.2 (FIG. 7C-7D).

### Example 4. Tumor Growth Inhibition by Anti-CLDN18.2-4-1BB Bispecific Antibodies

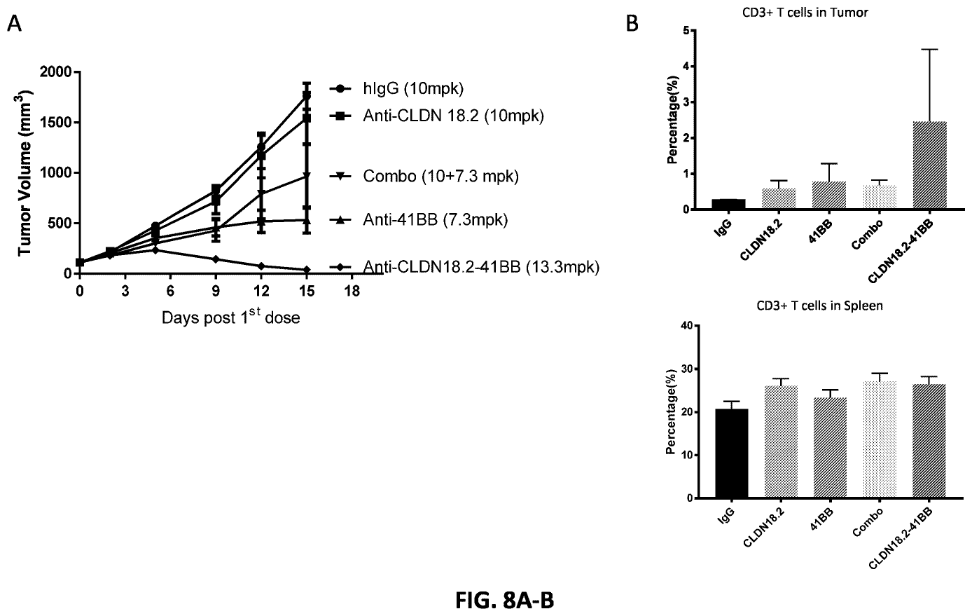

Humanized mice that expressed the extracellular domain of human 4-1BB were used. Mouse colon adenocarcinoma cells (MC38) were engineered to express human CLDN18.2. Humanized mice (h4-1BB) were subcutaneously implanted with MC38-hCLDN18.2 cells. Mouse were intraperitoneally administered every 3 days for 5 times with following antibodies: isotype control (10 mg/kg), anti-CLDN18.2 antibody (10 mg/kg), anti-4-1BB antibody (10 mg/kg), combination of anti-CLDN18.2 (10 mg/kg) and anti-4-1BB (10 mg/kg) and anti-CLDN18.2-4-1BB bispecific antibody (13.3 mg/kg). Tumor volumes were monitored by caliper measurement twice per week for the duration of the experiment. Tumor growth inhibition induced by bispecific antibodies was significantly greater than that observed with the combination of each targeting monoclonal antibodies, shown in FIG. 8. To further understand the mechanism of the antibody, tumor infiltrated lymphocytes and peripheral lymphocytes were collected and analyzed for the percentage of CD3+ T cells by flow cytometry. Results showed that bispecific antibodies can specifically increase the number of CD3+ T cells in the tumor microenvironment, while had no impact on peripheral blood.

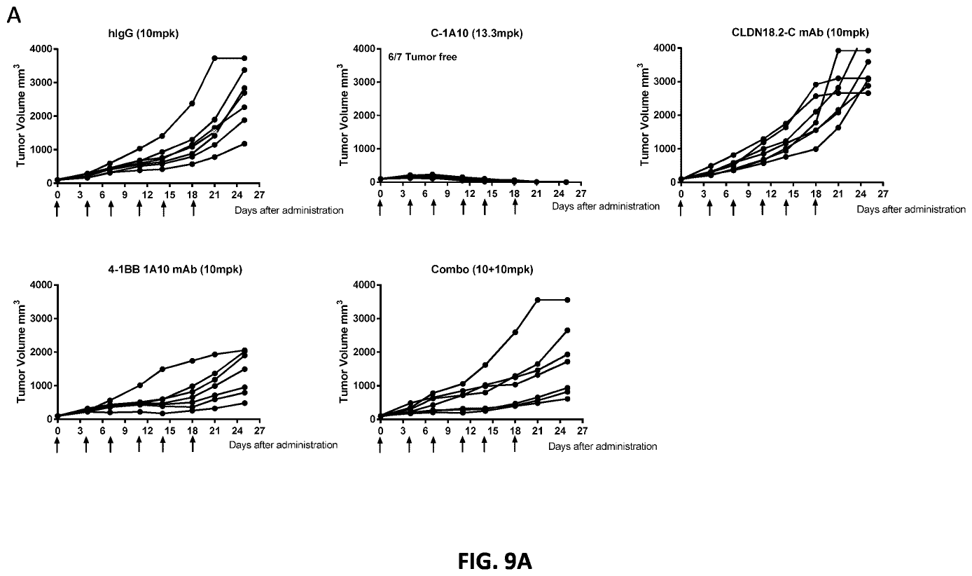

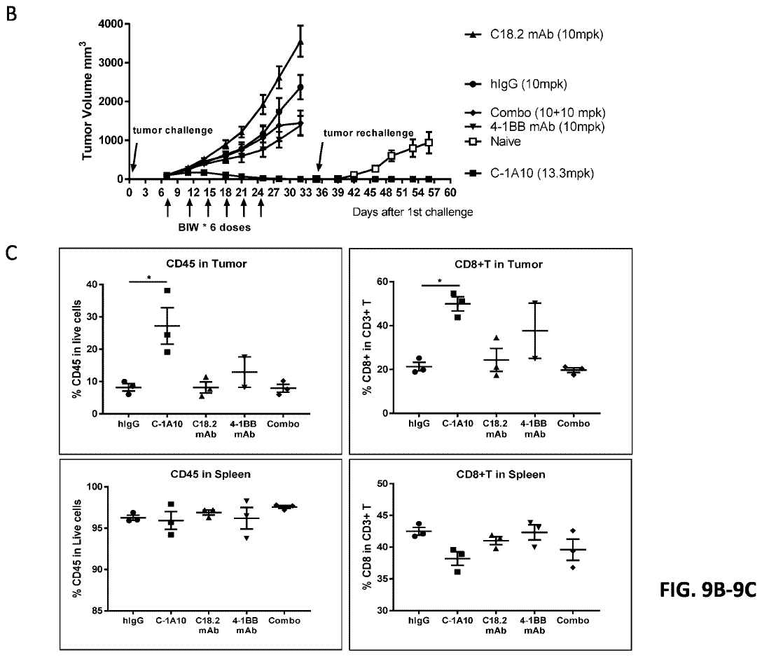

In another repeated experiment using the same animal model, the in vivo efficacy was further proved. The FIG. 9A showed the tumor growth curve of each animal in each group. The tumor growth inhibition of anti-CLDN18.2-4-1BB antibody (C-1A10) reached 105% at the end of study. Six of 7 mice in the group of bispecific were tumor free by day 25 following the first treatment.

In addition, this example re-challenged all anti-CLDN18.2-4-1BB antibody (C-1A10)-treated animals with a second dose of MC38-hCLDN18.2 tumor cells in the contralateral flank 35 days after the first tumor inoculation and monitored tumor growth without giving additional treatment. Results showed that as the tumor cells continued to grow in naïve mice, all BsAb-treated mice were resistant to tumor re-challenge and were deemed tumor free till the end of the study (FIG. 9B), suggesting that the present bispecific antibodies can induce long-term protective immunological memory again MC38 tumors. Similarly, in a satellite group (N=3/group) where mice received the same treatment, tumors were extracted 3 days after the second dose of antibodies to quantify tumor-infiltrating lymphocytes (TILs). The percentage of CD45+ and CD8+TILs was significantly higher in BsAb treatment group. In contrast, there was no effect on the peripheral lymphocytes (FIG. 9C).

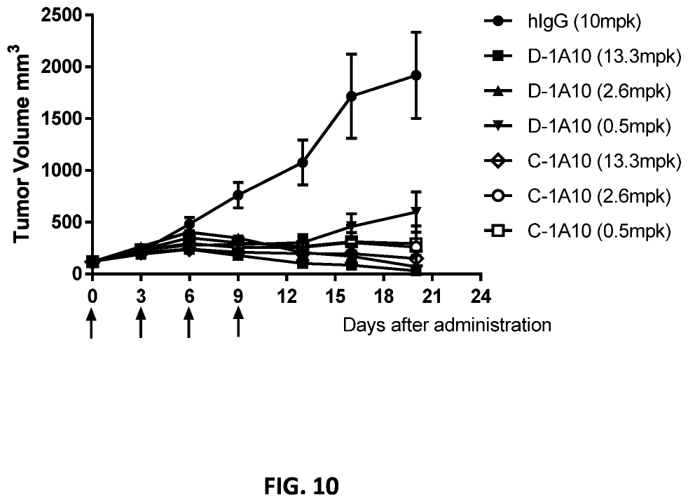

To further evaluate the anti-tumor efficacy of anti-CLDN18.2-4-1BB bispecific antibodies, different concentrations of bispecific antibodies were given to 4-1BB humanized mice implanted with MC38-hCLDN18.2 antibodies. Mouse were intraperitoneally administered every 3 days for 4 times with following antibodies: isotype control (10 mg/kg), C-1A10 (13.3 mg/kg), C-1A10 (2.6 mg/kg), C-1A10 (0.5 mg/kg), D-1A10 (13.3 mg/kg), D-1A10 (2.6 mg/kg), D-1A10 (0.5 mg/kg). Tumor volumes were monitored by caliper measurement twice per week for the duration of the experiment. As shown in FIG. 10, both C-1A10 and D-1A10 can suppress the tumor growth in a dose-dependent manner.

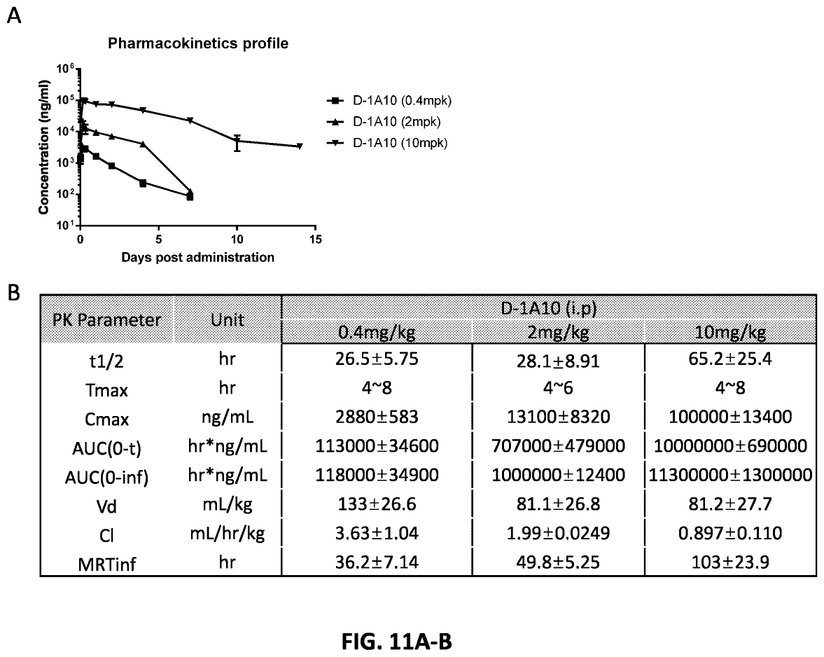

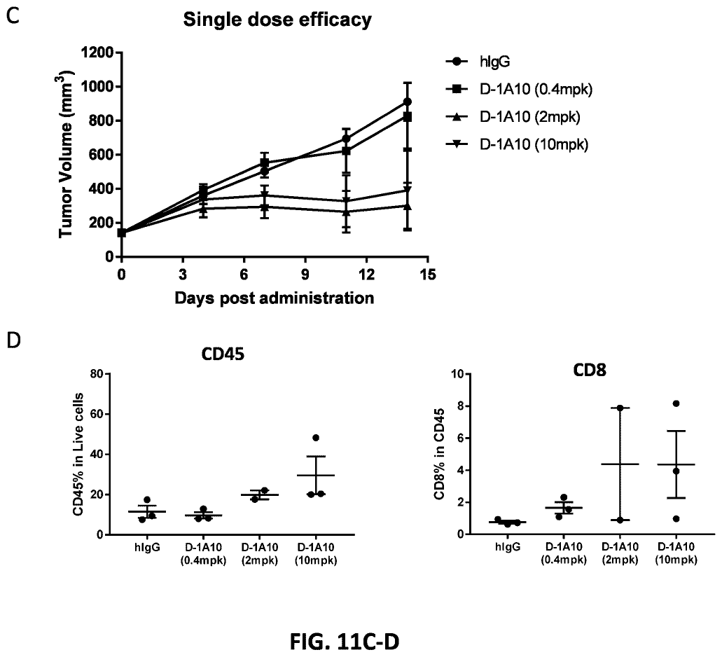

To understand the pharmacokinetics (PK) and pharmacodynamics relationship of anti-CLDN18.2-4-1BB in humanized mouse mode, single administration of D-1A10 at 3 different concentrations was given to tumor bearing-humanized mice. Serum concentration was measured at various time points. As showed in FIG. 11, overall, it displayed a dose-dependent PK profile. And the in vivo efficacy was correlated with the dose level. Ex vivo TIL analysis suggested a dose-dependent increase of CD8+ cells and CD45+ cells.

### Example 5. Testing of Additional Conditional Agonist Full Human Anti-4-1BB Antibodies

The following anti-4-1BB antibodies were identified for their ability to bind to 4-1BB at high affinity and not to activate 4-1BB signaling upon binding.

TABLE 4
Additional Anti-4-1BB Antibodies

| | | SEQ |
|---|---|---|
| Antibody | | ID |
| No. | VH | NO: |
| 41B01 | EVQLLESGGGLVQPGGSLRLSCAASGFTFSSYDMSWVRQAPGKGLEWVSWIS | 46 |
| | YSGGSIYYADSVKGRFTISRDNSKNTLYLQMNSLRAEDTAVYYCARDGQRNS | |
| | MREFDYWGQGTLVTVSS | |
| 41B01.01 | EVQLLESGGGLVQPGGSLRLSCAASGFTFSSYDMSWVRQAPGKGLEWVSWIS | 47 |
| | YSGGSIYYADSVKGRFTISRPNSKNTLYLQMNSLRAEPTAVYYCARDAQRNS | |
| | MREFDYWGQGTLVTVSS | |
| 41B01.02 | EVQLLESGGGLVQPGGSLRLSCAASGFTFSSYDMSWVRQAPGKGLEWVSWIS | 48 |
| | YSGGSIYYADSVKGRFTISRPNSKNTLYLQMNSLRAEPTAVYYCARDAQRQS | |
| | MREFDYWGQGTLVTVSS | |
| 41B01.03 | EVQLLESGGGLVQPGGSLRLSCAASGFTFSSYDMSWVRQAPGKGLEWVSWIS | 49 |
| | YSGGSIYYADSVKGRFTISRPNSKNTLYLQMNSLRAEPTAVYYCARDGQRQS | |
| | MREFDYWGQGTLVTVSS | |
| 41B01.04 | EVQLLESGGGLVQPGGSLRLSCAASGFTFSSYDMSWVRQAPGKGLEWVSWIS | 50 |
| | YSGGSIYYADSVKGRFTISRPNSKNTLYLQMNSLRAEPTAVYYCARDAQRNA | |
| | MREFDYWGQGTLVTVSS | |
| 41B02 | EVQLLESGGGLVQPGGSLRLSCAASGFTFSGYDMSWVRQAPGKGLEWVSVIY | 51 |
| | PDDGNTYYADSVKGRFTISRPNSKNTLYLQMNSLRAEPAAVYYCAKHGGQKP | |
| | TTKSSSAYGMDGWGQGTLVTVSS | |
| | | SEQ |
| Antibody | | ID |
| No. | VL | NO: |
| 41B01 | QSVLTQPPSASGTPGRRVTISCSGSSSNIGNNYVTWYQQLPGTAPKLLIYAD | 52 |
| | SHRPSGVPDRFSGSKSGTSASLAISGLRSEDEADYYCATWDYSLSGYVFGGG | |
| | TKLTVL | |
| 41B01.01 | QSVLTQPPSASGTPGRRVTISCSGSSSNIGNNYVTWYQQLPGTAPKLLIYAD | 52 |
| | SHRPSGVPDRFSGSKSGTSASLAISGLRSEDEADYYCATWDYSLSGYVFGGG | |
| | TKLTVL | |
| 41B01.02 | QSVLTQPPSASGTPGRRVTISCSGSSSNIGNNYVTWYQQLPGTAPKLLIYAD | 52 |
| | SHRPSGVPPRFSGSKSGTSASLAISGLRSEPEAPYYCATWDYSLSGYVFGGG | |
| | TKLTVL | |
| 41B01.03 | QSVLTQPPSASGTPGQRVTISCSGSSSNIGNNYVTWYQQLPGTAPKLLIYAD | 53 |
| | SHRPSGVPPRFSGSKSGTSASLAISGLRSEPEAPYYCATWDYSLSGYVFGGG | |
| | TKLTVL | |
| 41B01.04 | QSVLTQPPSASGTPGQRVTISCSGSSSNIGNNYVTWYQQLPGTAPKLLIYAD | 53 |
| | SHRPSGVPPRFSGSKSGTSASLAISGLRSEPEAPYYCATWDYSLSGYVFGGG | |
| | TKLTVL | |
| 41B02 | QSVLTQPPSASGTPGRRVTISCSGSSSNIGNNYVTWYQQLPGTAPKLLIYAD | 52 |
| | SHRPSGVPPRFSGSKSGTSASLAISGLRSEDEAPYYCATWDYSLSGYVFGGG | |
| | TKLTVL | |

CDRs for VHs in Table 4

| SEQ | | SEQ | | SEQ | |
|---|---|---|---|---|---|
| ID | | ID | | ID | |
| NO | VH_CDR1 | NO | VH_CDR2 | NO | VH_CDR3 |
| 1 | SYDMS | 2 | WISYSGGSIYYADSVKG | 3 | DAQRNSMREFDY |
| | | | | 56 | DGQRNSMREFDY |
| | | | | 57 | DAQRQSMREFDY |
| | | | | 58 | DGQRQSMREFDY |
| | | | | 59 | DAQRNAMREFDY |
| 60 | GYDMS | 61 | VIYPDDGNTYYADSVKG | 62 | HGGQKPTTKSSS |
| | | | | | AYGMDG |

TABLE 5
VHs and VLs of Anti-4-1BB scFv

| | | SEQ |
|---|---|---|
| Antibody | | ID |
| No. | VH | NO: |
| 41B01 | EVQLLESGGGLVQPGGSLRLSCAASGFTFSSYDMSWVRQAPGKCLEWVSWIS | 63 |
| | YSGGSIYYADSVKGRFTISRDNSKNTLYLQMNSLRAEDTAVYYCARDGQRNS | |
| | MREFDYWGQGTLVTVSS | |
| 41B01.01 | EVQLLESGGGLVQPGGSLRLSCAASGFTFSSYDMSWVRQAPGKCLEWVSWIS | 64 |
| | YSGGSIYYADSVKGRFTISRDNSKNTLYLQMNSLRAEDTAVYYCARDAQRNS | |
| | MREFDYWGQGTLVTVSS | |
| 41B01.03 | EVQLLESGGGLVQPGGSLRLSCAASGFTFSSYDMSWVRQAPGKCLEWVSWIS | 65 |
| | YSGGSIYYADSVKGRFTISRDNSKNTLYLQMNSLRAEDTAVYYCARDAQRNS | |
| | MREFDYWGQGTLVTVSS | |
| 41B01.04 | EVQLLESGGGLVQPGGSLRLSCAASGFTFSSYDMSWVRQAPGKCLEWVSWIS | 66 |
| | YSGGSIYYADSVKGRFTISRDNSKNTLYLQMNSLRAEDTAVYYCARDAQRQS | |
| | MREFDYWGQGTLVTVSS | |
| 41B02 | EVQLLESGGGLVQPGGSLRLSCAASGFTFSGYDMSWVRQAPGKCLEWVSVIY | 67 |
| | PDDGNTYYADSVKGRFTISRDNSKNTLYLQMNSLRAEDAAVYYCAKHGGQKP | |
| | TTKSSSAYGMDGWGQGTLVTVSS | |
| 41B02.01 | EVQLLESGGGLVQPGGSLRLSCAASGFTFSGYDMSWVRQAPGKCLEWVSVIY | 68 |
| | PDDGNTYYADSVKGRFTISRDNSKNTLYLQMNSLRAEDTAVYYCAKHGGQKP | |
| | TTKSSSAYGMDGWGQGTLVTVSS | |
| | | SEQ |
| Antibody | | ID |
| No. | VL | NO: |
| 41B01 | QSVLTQPPSASGTPGRRVTISCSGSSSNIGNNYVTWYQQLPGTAPKLLIYAD | 69 |
| | SHRPSGVPDRFSGSKSGTSASLAISGLRSEDEADYYCATWDYSLSGYVFGCG | |
| | TKLTVL | |
| 41B01.01 | QSVLTQPPSASGTPGRRVTISCSGSSSNIGNNYVTWYQQLPGTAPKLLIYAD | 70 |
| | SHRPSGVPDRFSGSKSGTSASLAISGLRSEDEADYYCATWDYSLSGYVFGCG | |
| | TKLTVL | |
| 41B01.03 | QSVLTQPPSASGTPGQRVTISCSGSSSNIGNNYVTWYQQLPGTAPKLLIYAD | 71 |
| | SHRPSGVPDRFSGSKSGTSASLAISGLRSEDEADYYCATWDYSLSGYVFGCG | |
| | TKLTVL | |
| 41B01.04 | QSVLTQPPSASGTPGQRVTISCSGSSSNIGNNYVTWYQQLPGTAPKLLIYAD | 72 |
| | SHRPSGVPDRFSGSKSGTSASLAISGLRSEDEADYYCATWDYSLSGYVFGCG | |
| | TKLTVL | |
| 41B02 | QSVLTQPPSASGTPGRRVTISCSGSSSNIGNNYVTWYQQLPGTAPKLLIYAD | 73 |
| | SHRPSGVPDRFSGSKSGTSASLAISGLRSEDEADYYCATWDYSLSGYVFGCG | |
| | TKLTVL | |
| 41B02.01 | QSVLTQPPSASGTPGQRVTISCSGSSSNIGNNYVTWYQQLPGTAPKLLIYAD | 74 |
| | SHRPSGVPDRFSGSKSGTSASLAISGLRSEDEADYYCATWDYSLSGYVFGCG | |
| | TKLTVL | |

### 5.1 Antigen Binding Measured by ELISA

To evaluate the antigen binding activity, the antibodies were subjected to ELISA test. Briefly, microtiter plates were coated with human 4-1BB-Fc protein at 0.1 μg/ml in PBS, 100 μl/well at 4° C. overnight, then blocked with 100 μl/well of 5% BSA. Five-fold dilutions of the antibodies starting from 10 g/ml were added to each well and incubated for 1-2 hours at RT. The plates were washed with PBS/Tween and then incubate with goat-anti-human IgG antibody conjugated with Horse Radish Peroxidase (HRP) for 1 hour at RT. After washing, the plates were developed with TMB substrate and analyzed by spectrophotometer at OD 450-630 nm. The anti-4-1BB antibodies tested showed 4-1BB binding abilities.

### 5.2 Cell Binding Measured by FACS

To evaluate the antigen binding property, the antibody candidates were analyzed for its binding to mammalian expressed 4-1BB by FACS. Briefly, 4-1BB-Jurkat cells were incubated with the antibodies. After wash by FACS buffer (1% BSA in PBS), the FITC-anti-human IgG antibody was added to each well and incubated at 4° C. for 1 hour. The MFI of FITC was evaluated by FACS Caliber. The anti-4-1BB antibodies tested showed binding abilities to 4-1BB which expressed on cell surface and can efficiently bind to 4-1BB expressed on mammalian cells.

### 5.3 Protein Kinetic for 4-1BB

To explore the binding kinetics of the antibodies, this example performed the affinity ranking by using Octet Red 96. As shown in Table 5, the anti-4-1BB antibodies tested had high 4-1BB binding affinities.

TABLE 5
Binding Kinetics

| Antibody | KD (M) | kon(1/Ms) | kdis(1/s) | Chi | R² |
|---|---|---|---|---|---|
| 41B01 | 1.80E−10 | 6.58E+05 | 1.19E−04 | 0.0392 | 0.9987 |
| 41B02 | 1.01E−09 | 5.95E+05 | 6.03E−04 | 0.0525 | 0.9973 |

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An antibody, comprising:
an anti-claudin 18.2 (CLDN18.2) unit having binding specificity to a CLDN18.2 protein; and
an anti-4-1BB unit having binding specificity to a 4-1BB protein,
wherein the anti-4-1BB unit is incapable of activating 4-1BB signaling upon binding to a 4-1BB protein, in the absence of the anti-CLDN18.2 unit binding to a CLDN18.2 protein.

2. The antibody of claim 1, wherein the binding of the anti-4-1BB unit to 4-1BB proteins on a cell does not result in clustering of the 4-1BB proteins, in the absence of the anti-CLDN18.2 unit binding to a CLDN18.2 protein.

3. The antibody of claim 1 or 2, further comprising a Fc fragment that has reduced effector function.

4. The antibody of claim 3, wherein the effector function is antibody-dependent cell-mediated cytotoxicity (ADCC), complement-dependent cytotoxicity (CDC), or antibody-dependent cellular phagocytosis (ADCP).

5. The antibody of claim 4, wherein the Fc fragment has reduced binding or no binding to FcγR or C1q.

6. The antibody of claim 5, wherein the Fc fragment has one or more mutation to reduce or eliminate FcγR or C1q binding.

7. The antibody of claim 6, wherein the Fc fragment is selected from the group consisting of:
an IgG1 Fc fragment with a L235E mutation,
an IgG1 Fc fragment with a L234A and/or L235A mutation,
an IgG1 Fc fragment with a P329G or P329A mutation,
an IgG4 Fc fragment with a F234A and/or L235A or L235E mutation,
an IgG2 Fc fragment with a H268Q, V309L, A330S, and/or P331S mutation, and
an IgG2 Fc fragment with a V234A, G237A, P238S, H268A, V309L, A330S, and/or P331S mutation (EU numbering).

8. The antibody of claim 3, wherein the Fc fragment is aglycosylated.

9. The antibody of claim 8, wherein the Fc fragment comprises a mutation that eliminates glycosylation.

10. The antibody of claim 9, wherein the mutation is at N297, optionally N297A, N297G, or N297Q.

11. The antibody of claim 3, wherein the Fc fragment comprises a mutation of N297A or a combination of mutations of L234A and L235A.

12. The antibody of any one of claims 1-11, wherein the anti-CLDN18.2 unit comprises a Fab fragment.

13. The antibody of claim 12, wherein the anti-CLDN18.2 unit comprises a pair of Fab fragment.

14. The antibody of any one of claims 1-13, wherein the anti-4-1BB unit comprises a Fab fragment or a single chain fragment (scFv).

15. The antibody of claim 14, wherein the anti-4-1BB unit comprises a pair of the scFv.

16. The antibody of claim 14 or 15, wherein the scFv is at the C-terminal side of the Fc fragment of the anti-CLDN18.2 unit.

17. The antibody of any one of claims 1-16, wherein the anti-4-1BB unit comprises a heavy chain variable region (VH) comprising a CDRH1, a CDRH2, and a CDRH3, and a light chain variable region (VL) comprising a CDRL1, a CDRL2 and a CDRL3, wherein:
the CDRH1 comprises the amino acid sequence of SEQ ID NO:1, or an amino acid sequence having one or two amino acid substitution from SEQ ID NO:1;
the CDRH2 comprises the amino acid sequence of SEQ ID NO:2, or an amino acid sequence having one or two amino acid substitution from SEQ ID NO:2;
the CDRH3 comprises the amino acid sequence of SEQ ID NO:3, 56, 57, 58, or 59, or an amino acid sequence having one or two amino acid substitution from SEQ ID NO: 3, 56, 57, 58, or 59;
the CDRL1 comprises the amino acid sequence of SEQ ID NO:4 or 60, or an amino acid sequence having one or two amino acid substitution from SEQ ID NO:4 or 60;
the CDRL2 comprises the amino acid sequence of SEQ ID NO:5 or 61, or an amino acid sequence having one or two amino acid substitution from SEQ ID NO:5 or 61; and
the CDRL3 comprises the amino acid sequence of SEQ ID NO:6 or 62, or an amino acid sequence having one or two amino acid substitution from SEQ ID NO:6 or 62.

18. The antibody of any one of claims 1-16, wherein the anti-4-1BB unit comprises a heavy chain variable region (VH) comprising a CDRH1, a CDRH2, and a CDRH3, and a light chain variable region (VL) comprising a CDRL1, a CDRL2 and a CDRL3, wherein:
the CDRH1 comprises the amino acid sequence of SEQ ID NO:1;
the CDRH2 comprises the amino acid sequence of SEQ ID NO:2;
the CDRH3 comprises the amino acid sequence of SEQ ID NO:3, 56, 57, 58, or 59;
the CDRL1 comprises the amino acid sequence of SEQ ID NO:4 or 60;
the CDRL2 comprises the amino acid sequence of SEQ ID NO:5 or 61; and
the CDRL3 comprises the amino acid sequence of SEQ ID NO:6 or 62.

19. The antibody of any one of claims 1-18, wherein the anti-4-1BB unit comprises a heavy chain variable region (VH) comprising an amino acid sequence selected from the group consisting of SEQ ID NO:24, 46-51 and 63-68 and a light chain variable region (VL) comprising an amino acid sequence selected from the group consisting of SEQ ID NO:25, 52-53 and 69-74.

20. The antibody of any one of claims 1-19, wherein the anti-CLDN18.2 unit comprises a heavy chain variable region (VH) comprising a CDRH1, a CDRH2, and a CDRH3, and a light chain variable region (VL) comprising a CDRL1, a CDRL2 and a CDRL3, wherein:
(a) the CDRH1 comprises the amino acid sequence of SEQ ID NO:7;
the CDRH2 comprises the amino acid sequence of SEQ ID NO:8;
the CDRH3 comprises the amino acid sequence of SEQ ID NO:9;
the CDRL1 comprises the amino acid sequence of SEQ ID NO:10;
the CDRL2 comprises the amino acid sequence of SEQ ID NO:11; and
the CDRL3 comprises the amino acid sequence of SEQ ID NO:12,
(b) the CDRH1 comprises the amino acid sequence of SEQ ID NO:13;
the CDRH2 comprises the amino acid sequence of SEQ ID NO:14;
the CDRH3 comprises the amino acid sequence of SEQ ID NO:15;
the CDRL1 comprises the amino acid sequence of SEQ ID NO:16;
the CDRL2 comprises the amino acid sequence of SEQ ID NO:17; and
the CDRL3 comprises the amino acid sequence of SEQ ID NO:18, or
(c) the CDRH1 comprises the amino acid sequence of SEQ ID NO:19;
the CDRH2 comprises the amino acid sequence of SEQ ID NO:20;
the CDRH3 comprises the amino acid sequence of SEQ ID NO:21;
the CDRL1 comprises the amino acid sequence of SEQ ID NO:22;
the CDRL2 comprises the amino acid sequence of SEQ ID NO:11; and
the CDRL3 comprises the amino acid sequence of SEQ ID NO:23.

21. The antibody of claim 20, wherein the VH of the anti-CLDN18.2 unit comprises an amino acid sequence selected from the group consisting of SEQ ID NO:26, 28 and 30, and the VL of the anti-CLDN18.2 unit comprises an amino acid sequence selected from the group consisting of SEQ ID NO:27, 29 and 31.

22. An antibody comprising two first polypeptides and two second polypeptides,
wherein each first polypeptide comprises, from N- to C-terminus, a heavy chain variable region (VH), a CHI, a CH2, a CH3, and a single chain fragment (scFv) having specificity to a 4-1BB protein;
wherein each second polypeptide comprises a light chain variable region (VL) and a CL;
wherein each VH is paired with one of the VL and have specificity to a claudin 18.2 (CLDN18.2) protein; and
wherein the scFv is incapable of activating 4-1BB signaling upon binding to a 4-1BB protein, in the absence of the VH/VL pairs binding to a CLDN18.2 protein.

23. The antibody of claim 22, wherein the CH2 and CH3 constitute an Fc fragment that has reduced effector function.

24. The antibody of claim 23, wherein the Fc fragment is selected from the group consisting of:
an IgG1 Fc fragment with a L235E mutation,
an IgG1 Fc fragment with a L234A and/or L235A mutation,
an IgG1 Fc fragment with a P329G or P329A mutation,
an IgG4 Fc fragment with a F234A and/or L235A or L235E mutation,
an IgG2 Fc fragment with a H268Q, V309L, A330S, and/or P331S mutation, and
an IgG2 Fc fragment with a V234A, G237A, P238S, H268A, V309L, A330S, and/or P331S mutation (EU numbering).

25. The antibody of claim 23, wherein the Fc fragment comprises a mutation at N297, optionally N297A, N297G, or N297Q.

26. The antibody of any one of claims 22-25, wherein the scFv comprises a heavy chain variable region (VH) comprising a CDRH1, a CDRH2, and a CDRH3, and a light chain variable region (VL) comprising a CDRL1, a CDRL2 and a CDRL3, wherein:
the CDRH1 comprises the amino acid sequence of SEQ ID NO:1;
the CDRH2 comprises the amino acid sequence of SEQ ID NO:2;
the CDRH3 comprises the amino acid sequence of SEQ ID NO: 3, 56, 57, 58, or 59;
the CDRL1 comprises the amino acid sequence of SEQ ID NO:4 or 60;
the CDRL2 comprises the amino acid sequence of SEQ ID NO:5 or 61; and
the CDRL3 comprises the amino acid sequence of SEQ ID NO:6 or 62.

27. The antibody of any one of claims 22-26, wherein the scFv comprises a heavy chain variable region (VH) comprising an amino acid sequence selected from the group consisting of SEQ ID NO:24, 46-51 and 63-68 and a light chain variable region (VL) comprising an amino acid sequence selected from the group consisting of SEQ ID NO:25, 52-53 and 69-74.

28. The antibody of any one of claims 22-27, wherein the VH of the VH/VL pair comprises a CDRH1, a CDRH2, and a CDRH3, and VL of the VH/VL pair comprises a CDRL1, a CDRL2 and a CDRL3, wherein:
(a) the CDRH1 comprises the amino acid sequence of SEQ ID NO:7;
the CDRH2 comprises the amino acid sequence of SEQ ID NO:8;
the CDRH3 comprises the amino acid sequence of SEQ ID NO:9;
the CDRL1 comprises the amino acid sequence of SEQ ID NO:10;
the CDRL2 comprises the amino acid sequence of SEQ ID NO:11; and
the CDRL3 comprises the amino acid sequence of SEQ ID NO:12,
(b) the CDRH1 comprises the amino acid sequence of SEQ ID NO:13;
the CDRH2 comprises the amino acid sequence of SEQ ID NO:14;
the CDRH3 comprises the amino acid sequence of SEQ ID NO:15;
the CDRL1 comprises the amino acid sequence of SEQ ID NO:16;
the CDRL2 comprises the amino acid sequence of SEQ ID NO:17; and
the CDRL3 comprises the amino acid sequence of SEQ ID NO:18, or
(c) the CDRH1 comprises the amino acid sequence of SEQ ID NO:19;
the CDRH2 comprises the amino acid sequence of SEQ ID NO:20;
the CDRH3 comprises the amino acid sequence of SEQ ID NO:21;
the CDRL1 comprises the amino acid sequence of SEQ ID NO:22;
the CDRL2 comprises the amino acid sequence of SEQ ID NO:11; and
the CDRL3 comprises the amino acid sequence of SEQ ID NO:23.

29. The antibody of claim 27, wherein the VH of the VH/VL pair comprises an amino acid sequence selected from the group consisting of SEQ ID NO:26, 28 and 30, and the VL of the VH/VL pair comprises an amino acid sequence selected from the group consisting of SEQ ID NO:27, 29 and 31.

30. The antibody of claim 22, wherein each of the first polypeptides comprises the amino acid sequence of SEQ ID NO:40 and each of the second polypeptides comprises the amino acid sequence of SEQ ID NO:41.

31. The antibody of claim 22, wherein each of the first polypeptides comprises the amino acid sequence of SEQ ID NO:42 and each of the second polypeptides comprises the amino acid sequence of SEQ ID NO:43.

32. The antibody of claim 22, wherein each of the first polypeptides comprises the amino acid sequence of SEQ ID NO:44 and each of the second polypeptides comprises the amino acid sequence of SEQ ID NO:45.

33. One or more polynucleotide encoding the antibody of any one of claims 1-32.

34. A composition comprising the antibody of any one of claims 1-32 and a pharmaceutically acceptable carrier.

35. Use of the antibody of any one of claims 1-32 for the preparation of a medicament for the treatment of cancer.

36. A method for treating cancer in a patient in need thereof, comprising administering to the patient the antibody of any one of claims 1-32.

37. The use of claim 35 or the method of claim 36, wherein the cancer is characterized by a cancer cell that overexpresses CLDN18.2 as compared to a counterpart normal cell.

38. The use of claim 35 or 37, or the method of claim 36 or 37, wherein the cancer is an epithelial tumor, or wherein the cancer is selected from the group consisting of bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer.

39. The use of claim 35 or 37, or the method of claim 36 or 37, wherein the cancer is selected from the group consisting of gastric cancer, pancreatic cancer, esophageal cancer, lung cancer and ovarian cancer.
